(19) United States
(12) Patent Application Publication
(10) Pub. No.: US 2004/0023848 A1
(43) Pub. Date: Feb. 5, 2004

(54) Compositions for the treatment, prevention, and diagnosis of gastrointestinal and other infections

(76) Inventor: Thomas Boehm, Brookline, MA (US)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/375,690

(22) Filed: Feb. 27, 2003

Related U.S. Application Data
(60) Provisional application No. 60/359,831, filed on Feb. 27, 2002.

(51) Int. Cl. A61K 38/00; A61K 31/715; A61K 9/14

(52) U.S. Cl. 514/2; 424/489; 514/44; 514/54

(57) ABSTRACT

The present invention relates to pharmaceutical compositions that bind or kill gastrointestinal and other microorganisms, as well as methods of making and using the same.

### BACKGROUND OF THE INVENTION

Drug resistance by microbes (e.g., bacteria, viruses, parasites, and fungi) is a global clinical and public health problem that has emerged with alarming rapidity in recent years and undoubtedly will increase in the near future. Resistance is a problem in the community as well as in health care settings, where transmission of such microbes is greatly amplified. Because multiple drug resistance is a growing problem, physicians are now confronted with infections for which there is no effective therapy. The morbidity, mortality, and financial costs of such infections pose an increasing burden for health care systems worldwide, but especially in countries with limited resources. Strategies to address these issues have emphasized enhanced surveillance of drug resistance, increased monitoring and improved usage of antimicrobial drugs, professional and public education, development of new drugs, and assessment of alternative therapeutic modalities.

Inherent to all current approaches is the therapeutic goal of destroying or interrupting the replication of the target microorganism. Although the sequencing of microbial genomes makes it easier to find adequate targets, there is a general problem with this approach. Viruses, parasites, fungi, and bacteria have short replication cycles and highly variable genetic elements, which allow the selection of resistant strains. In particular, if the susceptible non-pathogenic microbes of the normal flora in a subject are suppressed or even killed by a therapeutic agent, resistant pathogenic strains have a considerable survival advantage because additional nutrients and other resources become available. Thus, current treatment methods facilitate the Darwinian selection of resistant microorganisms. Alternative and improved agents are needed for the treatment of microbial infections that do not facilitate this selection.

Various microbial infections of the gastrointestinal tract affect hundreds of millions of people per year. Many of these infections are caused by foodborne or waterborne microbes, such as Salmonella bacteria, E. coli bacteria, Norwalk viruses, and Giardia lamblia parasites. Other infections, such as those by Clostridrium difficile and vancomycin-resistant Enterococci, are caused by the heavy antibiotic therapy to which patients in intensive care units are subjected. Still other infections occur as septicemias in immunocompromised patients, such as those by Candida albicans, parapsilosis, and glabrata. Finally, many other communicable gastrointestinal infections are known, such as Helicobacter pylori infection, which is the major cause of gastric and duodenal ulcers, and Rotavirus infection, the most important etiologic agent of infantile gastroenteritis. Many of these gastrointestinal infection-causing agents have already developed resistant strains, or “superbugs”, in response to conventional drug treatment. For some others, there is still no effective conventional therapeutic agent or vaccine available.

Therefore, there is a need for novel approaches to the prevention and treatment of microbe-induced gastrointestinal disorders.

### SUMMARY OF THE INVENTION

In part, the present invention is directed to pharmaceutical compositions that bind gastrointestinal microorganisms after being ingested by a subject and are then cleared from the gastrointestinal tract. The non-absorbable compositions bind specifically to the target microorganisms and do not remove the normal flora, thus reducing or eliminating the conditions that facilitate the development of microbial resistance. Because the compositions have an extracellular mode of action, they are expected to be safe. Furthermore, because multiple receptors may be targeted in a single composition of the present invention, the composition may be made serotype-independent or may be able to bind more than one species of microorganism. The present invention is also directed to methods of making said compositions. The compositions may be adapted for use in treating infections by other types of microorganisms, particularly those in other orifices of the body from which the compositions can be cleared.

In one embodiment of the present invention, the non-absorbable compositions are comprised of a particle of the following formula:

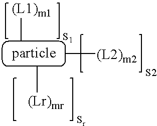

wherein;

L1, L2 . . . Lr, each independently, represent a molecule that is able to bind or kill a gastrointestinal or other microorganism; wherein said molecule is selected from the group consisting of lipids, carbohydrates, peptides, peptidomimetics, peptide-nucleic acids (PNAs), proteins, small molecules, natural products, aptamers and oligonucleotides; and wherein each Lr optionally may be conjugated to said particle through a linker molecule;

mr, independently for each Lr, is at least 1;

sr, independently for each Lr, is at least 1;

and r is at least 2.

In another embodiment of the invention, the non-absorbable compositions are comprised of a particle of the following formula:

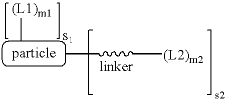

wherein;

L1 and L2 are as defined above; wherein L1 optionally may be conjugated to said particle through a linker molecule of the same type or a different type than the linker through which L2 is conjugated to said particle;

mr, independently for each Lr, is at least 1; and

sr, independently for each Lr, is at least 1.

In one embodiment of the invention, the non-absorbable compositions are comprised of a particle of the following formula:

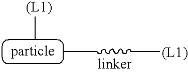

wherein;

L1 is as defined above; wherein some L1 optionally may be conjugated to said particle through a linker molecule of a different type than the linker through which other L1 are conjugated to said particle; and

at least two different L1-particle linkages are present.

In another embodiment of the invention, the a non-absorbable compositions are comprised of particle of the following formula:

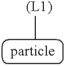

wherein;

L1 is as defined above; wherein L1 optionally may be conjugated to said particle through a linker molecule; and

said particle is able to release biologically active agents, contains agents able to kill a target microorganism, or is otherwise able to aid in the binding or killing of the target microorganism.

In another embodiment of the present invention, the non-absorbable compositions are comprised of a particle of the following formula:

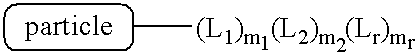

wherein;

L1, L2 . . . Lr, are as previously defined; wherein multiple Lr are conjugated to the particle through a single point of attachment, optionally through a linker molecule, and may be arranged in any order;

r is at least 2; and

mr, independently for each Lr, is at least 1.

The Lr in the above formula may be attached to each other sequentially in any order (at random or in blocks), optionally with linker molecules between them. Alternatively, the Lr may be attached to the conjugating moiety via a branched molecule, as in the following exemplary formula:

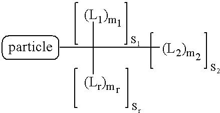

wherein;

L1, L2 . . . Lr, are as previously defined;

each Lr may be linked to the conjugating moiety through the same or different linkages;

r is at least 2;

s2, independently for each Lr, is at least 1; and

mr, independently for each Lr, is at least 1.

The particles may be comprised of at least one polymeric molecule. In certain embodiments, said molecule is polystyrene. In other embodiments, said molecule is a controlled release polymer. In certain of these embodiments, the controlled release polymer may optionally have a biologically active agent encapsulated within it or distributed throughout it. In certain embodiments, said particle is a bead and has a diameter in the range of about 1 to about 50 microns. In certain embodiments, the particle may be a macromolecular polymeric compound. In any of the embodiments, the particle may be comprised of multiple polymeric molecules.

The particles may be administered alone or in conjunction with a biologically active agent. In certain embodiments, a biologically active agent comprises the particle of the subject composition. A number of biologically active agents are contemplated for use with the present invention. A particle that contains a releasable biologically active agent may be comprised of a controlled release polymer or another material that allows the release of the agent. In certain embodiments, other materials may be added to the particles alone or in addition to the biologically active agent, to alter the physical and chemical properties of the resulting composition, including for example, the release profile of the agent from the particle. Examples of such materials include biocompatible plasticizers, delivery agents, fillers and the like.

The particles of the disclosed compositions may be conjugated through a linker molecule to at least one Lr molecule, or may be bound directly to at least one Lr molecule. In one embodiment of the invention, the particles of the composition are derivatized and conjugated to an Lr molecule simultaneously. In this embodiment, the chemical link between the derivatized chemical group of the particle and an Lr molecule serves as the linker. In another embodiment, the particles of the composition are derivatized, reacted with at least one additional linker molecule, and conjugated to an Lr molecule. In certain embodiments, the linker molecule may be a branched or linear polymer. In embodiments wherein said linker is branched, an Lr molecule may be present at the end of each branch. In certain embodiments of the invention, the linker is comprised of a polypeptide. In one embodiment of the invention, the linker is comprised of polyglycine. In other embodiments of the invention, the linker is comprised of both polyethylene glycol and polypropylene.

In certain embodiments of the invention the target microorganism of the subject composition is selected from the group consisting of bacteria, viruses, parasites, and fungi. In certain embodiments of the invention, the target microorganisms are bound to the composition via an Lr molecule. Lr molecules for use in the compositions may be selected by screening the ability of candidate molecules to bind said target microoorganism or molecule derived therefrom using appropriate assays as known to one of skill in the art. In one embodiment of the invention, candidate Lr molecules are selected from a library of molecules, which may optionally be synthethized through combinatorial methods. In still other embodiments of the invention, the target microorganisms are killed by an Lr molecule. In certain embodiments of the invention, a composition may contain both Lr molecules that bind and Lr molecules that kill a target microorganism. In other embodiments, a composition may contain several Lr that each bind a different target microorganism, or, alternatively, each bind a different surface molecule on the same microorganism.

By combining different Lr molecules linked to the particles via different chemical moieties, a composition may be able to more effectively bind or kill one or more target microorganisms than could a composition comprising a particle conjugated to a single ligand using identical moieties. Any combination of Lr molecules, conjugation chemistries, and/or linkers are contemplated for use in the present invention. The size, length, and physicochemical (e.g. distribution of functional groups, hydrophobicity, rigidity) properties of the linkers and ligands may be varied and combined onto a single subject particle. Such a variety of different ligands conjugated to a single particle, may, for instance, allow a ligand complex to be formed on the surface of the particle either before or after binding a target microorganism. For example, a complex of protein ligands (e.g. where Lr are proteins) on the surface of a particle could be used to bind a surface molecule of a microorganism.

The compositions of the present invention may also be adapted for use in treating microbial infection in other orifices in the body, for example, vaginal or oral fungal infection.

The present invention provides for pharmaceutical formulations of the compositions of the invention. In part, the subject invention is directed to preparations of formulations of compositions comprising a releasable biologically active agent. In another aspect, the subject compositions may be used in the manufacture of a medicament for any number of uses, including, for example, treating any gastrointestinal disease or other appropriate treatable condition of a patient.

In another aspect, the present invention is directed to methods of using the subject compositions for prophylactic, diagnostic, or therapeutic treatment of a colonization of gastrointestinal or other types of microorganisms in a patient. In embodiments where the treatment is therapeutic, said treatment may result in the decolonization of the gastrointestinal microorganisms. In certain embodiments, use of the subject that release a biologically active agent in a sustained manner allows for different treatment regimens than are possible with other modes of administration of such therapeutic agents. In embodiments where the use of the compositions is diagnostic, the compositions may be used to detect the presence of an infection by gastrointestinal microorganisms or track the efficacy of the treatment of an existing infection by gastrointestinal microorganisms in a patient.

The present invention includes a kit comprising subject compositions, and optionally instructions for their use. Uses for such kits include, for example, therapeutic, diagnostic, and prophylactic applications.

These embodiments of the present invention, other embodiments, and their features and characteristics, will be apparent from the description, drawings and claims that follow. Examples of such embodiments include those disclosed in appended claims, which are hereby incorporated by reference in their entirety into this Summary.

The practice of the present invention will employ, unless otherwise indicated, conventional techniques of chemistry, cell biology, cell culture, molecular biology, transgenic biology, microbiology, recombinant DNA, and medicine, which are within the skill of the art. Such techniques are explained fully in the literature.

### DETAILED DESCRIPTION OF THE INVENTION

1. Definitions

For convenience, before further description of the present invention, certain terms employed in the specification, examples and appended claims are defined here.

The singular forms “a”, “an”, and “the” include plural references unless the context clearly dictates otherwise.

The term “agonist” is art-recognized and refers to a compound that mimics the action of natural transmitter or, when the natural transmitter is not known, causes changes at the receptor complex in the absence of other receptor ligands.

The term “amino acid” is art-recognized and refers to all molecules, whether natural or synthetic, which include both an amino functionality and an acid functionality, including amino acid analogs and derivatives. In certain embodiments, the amino acids used in the application of this invention are those naturally occurring amino acids found in proteins, or the naturally occurring anabolic or catabolic products of such amino acids which contain amino and carboxyl groups. Particularly suitable amino acid side chains include side chains selected from those of the following amino acids: glycine, alanine, valine, cysteine, leucine, isoleucine, serine, threonine, methionine, glutamic acid, aspartic acid, glutamine, asparagine, lysine, arginine, proline, histidine, phenylalanine, tyrosine, and tryptophan.

The terms “amino acid residue” and “peptide residue” are art-recognized and refer to an amino acid or peptide molecule without the —OH of its carboxyl group. In general the abbreviations used herein for designating the amino acids and the protective groups are based on recommendations of the IUPAC-IUB Commission on Biochemical Nomenclature (see Biochemistry (1972) 11:1726-1732). For instance Met, Ile, Leu, Ala and Gly represent “residues” of methionine, isoleucine, leucine, alanine and glycine, respectively. By the residue is meant a radical derived from the corresponding α-amino acid by eliminating the OH portion of the carboxyl group and the H portion of the α-amino group. The term “amino acid side chain” is that part of an amino acid exclusive of the —CH(NH₂)COOH portion, as defined by Kopple, Peptides and Amino Acids 2, 33 (W. A. Benjamin Inc., New York and Amsterdam, 1966); examples of such side chains of the common amino acids are —CH₂CH₂SCH₃ (the side chain of methionine), —CH₂CH(CH₃)₂ (the side chain of leucine) or —H (the side chain of glycine).

The term “amino acid residue” further includes analogs, derivatives and congeners of any specific amino acid referred to herein, as well as C-terminal or N-terminal protected amino acid derivatives (e.g. modified with an N-terminal or C-terminal protecting group). For example, the present invention contemplates the use of amino acid analogs wherein a side chain is lengthened or shortened while still providing a carboxyl, amino or other reactive precursor functional group for cyclization, as well as amino acid analogs having variant side chains with appropriate functional groups. For instance, the subject molecules may include an amino acid analog such as, for example, cyanoalanine, canavanine, djenkolic acid, norleucine, 3-phosphoserine, homoserine, dihydroxy-phenylalanine, 5-hydroxytryptophan, 1-methylhistidine, 3-methylhistidine, diaminopimelic acid, omithine, or diaminobutyric acid. Other naturally occurring amino acid metabolites or precursors having side chains which are suitable herein will be recognized by those skilled in the art and are included in the scope of the present invention.

Also included are the (D) and (L) stereoisomers of such amino acids when the structure of the amino acid admits of stereoisomeric forms. The configuration of the amino acids and amino acid residues herein are designated by the appropriate symbols (D), (L) or (DL), furthermore when the configuration is not designated the amino acid or residue can have the configuration (D), (L) or (DL). It will be noted that the structure of some of the molecules of this invention includes asymmetric carbon atoms. It is to be understood accordingly that the isomers arising from such asymmetry are included within the scope of this invention. Such isomers may be obtained in substantially pure form by classical separation techniques and by sterically controlled synthesis. For the purposes of this application, unless expressly noted to the contrary, a named amino acid shall be construed to include both the (D) or (L) stereoisomers. In the majority of cases, D- and L-amino acids have R- and S-absolute configurations, respectively.

The names of the natural amino acids are abbreviated herein in accordance with the recommendations of IUPAC-IUB.

The term “antagonist” is art-recognized and refers to a compound that binds to a receptor site, but does not cause any physiological changes unless another receptor ligand is present.

The terms “antibiotic”, “germicide” and “antimicrobial” are art-recognized and refer to agents or molecules capable of killing, inactivating, or otherwise neutralizing the pathogenic or reproductive ability of microorganisms. Terms such as “bacteriocides”, “viricides” or “antivirals”, “antifungals”, “antihelmintics” and the like refer to categories of such agents which have the ability to kill, inactivate, or otherwise neutralize the pathogenic or reproductive ability of bacteria, viruses, fungi, and various parasites, respectively.

The term “antibody” is art-recognized and refers to whole antibodies, e.g., of any isotype (IgG, IgA, IgM, IgE, etc.), and includes fragments thereof which are also specifically reactive with a vertebrate, e.g., mammalian, protein. Antibodies may be fragmented using conventional techniques and the fragments screened for utility in the same manner as described above for whole antibodies. Thus, the term includes segments of proteolytically-cleaved or recombinantly-prepared portions of an antibody molecule that are capable of selectively reacting with a certain protein. Non-limiting examples of such proteolytic and/or recombinant fragments include Fab, F(ab′)2, Fab′, Fv, and single chain antibodies (scFv) containing a V[L] and/or V[H] domain joined by a peptide linker. The scfv's may be covalently or non-covalently linked to form antibodies having two or more binding sites. The subject invention includes polyclonal, monoclonal or other purified preparations of antibodies and recombinant antibodies. The term “protein” as applied to potential Lr molecules of the present invention includes “antibodies” as defined herein. “Human monoclonal antibodies” or “humanized” murine antibodies, as the terms are used herein, refer to whole murine monoclonal antibodies (or fragments thereof) “humanized” by genetically recombining the nucleotide sequence encoding the murine Fv region (i.e., containing the antigen binding site) or the complementarity-determining regions thereof with the nucleotide sequence encoding at least a human constant domain region and an Fc region, e.g., in a manner similar to that disclosed in European Patent Application Publication No. 0,411,893 A3. Some additional murine residues may also be retained within the human variable region framework domains to ensure proper target site binding characteristics. In certain embodiments, humanized antibodies may decrease the immunoreactivity of the antibody or polypeptide in the host recipient, permitting an increase in the half-life and a reduction in the possibility of adverse immune reactions.

A “bead” refers to any particle that is somewhat globular in shape, e.g. spherical, oblong, ellipsoidal, or droplike.

To “bind” or “interact” refers to include detectable interactions between molecules, such as may be detected using, for example, a hybridization assay. The term also includes “binding” interactions between molecules. Interactions may be, for example, protein-protein, protein-nucleic acid, protein-small molecule or small molecule-nucleic acid in nature. “Binding” may also refer to the interaction between a particle of the present invention and a microorganism. For example, a particle may “bind” a microorganism through an interaction between a molecule covalently linked to the particle and a molecule on the surface of the microorganism.

The term “biologically active agent” includes without limitation, medicaments; vitamins; mineral supplements; substances used for the treatment, prevention, diagnosis, cure or mitigation of a disease or illness; substances which affect the structure or function of the body or a cell or a microorganism; or pro-drugs, which become biologically active or more active after they have been placed in a predetermined physiological environment. The active substance(s) may be described as a single entity or a combination of entities. In addition, the term “biologically active agent” includes pharmaceutically acceptable salts of a biologically active agent, such as a hydrochloride salt. Non-limiting examples of biologically active agents are described in section 3.3.3.

The term “competitive antagonist” is art-recognized and refers to a compound that binds to a receptor site; its effects may be overcome by increased concentration of the agonist.

The term “decolonization” refers to the act or process of physically removing microorganisms from an area in which they have been established or are in the process of becoming established.

“Derived from” as that phrase is used herein indicates a peptide or nucleotide sequence selected from within a given sequence. A peptide or nucleotide sequence derived from a named sequence may contain a small number of modifications relative to the parent sequence, in most cases representing deletion, replacement or insertion of less than about 15%, preferably less than about 10%, and in many cases less than about 5%, of amino acid residues or base pairs present in the parent sequence. In the case of DNAs, one DNA molecule is also considered to be derived from another if the two are capable of selectively hybridizing to one another.

“Derivative” refers to the chemical modification of a polypeptide sequence, or a polynucleotide sequence. Chemical modifications of a polynucleotide sequence may include, for example, replacement of hydrogen by an alkyl, acyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

The term “disease” or “infection” refers to the establishment of a microorganism, such as a bacterium, virus, fungus, or parasite anywhere in a host, as well as the act or process of such establishment, and any resulting state in the host from said establishment, act, or process.

The term “ED₅₀” is art-recognized and refers to the dose of a drug or other compound or particle that is able to bind a gastrointestinal microorganism which produces 50% of its maximum response or effect, or alternatively, the dose which produces a pre-determined response in 50% of test subjects or preparations.

The term “gastrointestinal disease” or “gastrointestinal infection” refers to the establishment of a microorganism, such as a bacterium, virus, fungus, or parasite anywhere in a host's gastrointestinal tract, as well as the act or process of such establishment, and any resulting state in the host from said establishment, act, or process. Non-limiting examples of gastrointestinal diseases include cholera, colitis, dysentery, gastroenteritis, parasitic infections, ulcer and others. A “microorganism capable of producing a gastrointestinal disease or infection” is any microorganism that may establish itself in a host's gastrointestinal tract. Non-limiting examples of such microorganisms are described in section 3.6.

“Gene” or “recombinant gene” refer to a nucleic acid molecule comprising an open reading frame and including at least one exon and (optionally) an intron sequence. “Intron” refers to a DNA sequence present in a given gene which is spliced out during mRNA maturation.

“Gene construct” refers to a vector, plasmid, viral genome or the like which includes a “coding sequence” for a polypeptide or which is otherwise transcribable to a biologically active RNA (e.g., antisense, decoy, ribozyme, etc), may transfect cells, in certain embodiments mammalian cells, and may cause expression of the coding sequence in cells transfected with the construct. The gene construct may include one or more regulatory elements operably linked to the coding sequence, as well as intronic sequences, poly adenylation sites, origins of replication, marker genes, etc.

“Homology” or alternatively “identity” refers to sequence similarity between two peptides or between two nucleic acid molecules. Homology may be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. The term “percent identical” refers to sequence identity between two amino acid sequences, or between two nucleotide sequences. Identity may each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules may be referred to as homologous (similar) at that position. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and may be used with, e.g., default settings. ENTREZ is available through the National Center for Biotechnology Information, National Library of Medicine, National Institutes of Health, Bethesda, Md. In one embodiment, the percent identity of two sequences may be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method may be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences may be used to search both protein and DNA databases.

Databases with individual sequences are described in Methods in Enzymology, ed. Doolittle, supra. Databases include Genbank, EMBL, and DNA Database of Japan (DDBJ).

“Host cell” refers to a cell transduced with a specified transfer vector. The cell is optionally selected from in vitro cells such as those derived from cell culture, ex vivo cells, such as those derived from an organism, and in vivo cells, such as those in an organism. “Recombinant host cells” refers to cells which have been transformed or transfected with vectors constructed using recombinant DNA techniques. “Host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The term “LD₅₀” is art-recognized and refers to the dose of a drug or other compound or particle that is able to bind a gastrointestinal microorganism which is lethal in 50% of test subjects.

The terms “library” or “combinatorial library” refer to a plurality of molecules, which may be termed “members,” synthesized or otherwise prepared from one or more starting materials by employing either the same or different reactants or reaction conditions at each reaction in the library. In general, the members of any library show at least some structural diversity, which often results in chemical and biological diversity. Such structural diversity in preparing libraries of coordination molecules may include, by way of example, metal ion diversity, ligand diversity, solvation diversity or counter-ion diversity. A library may contain any number of members from two different members to about 108 members or more. In certain embodiments, libraries of the present invention have more than about 12, 50 and 90 members. In certain embodiments of the present invention, the starting materials and certain of the reactants are the same, and chemical diversity in such libraries is achieved by varying at least one of the reactants or reaction conditions during the preparation of the library. Combinatorial libraries of the present invention may be prepared in solution or on the solid phase. Further details regarding the libraries of the present invention are described below.

The term “linker” is art-recognized and refers to a molecule or group of molecules connecting a particle, including a bead or polymeric macromolecule, and an Lr molecule. A linker may also be a molecule or group of molecules connecting an Lr molecule to a particle conjugating moiety. The linker may be comprised of a single linking molecule or may comprise a linking molecule and a spacer molecule, intended to separate the linking molecule and the library member by a specific distance. Non-limiting examples of linkers for use in the present invention are described in section 3.4.

The term “microorganism” or “microbe” as used herein refers to any small entity capable of establishing itself within a subject, and includes bacteria, fungi, parasites, and viruses. The term “bacteria” refers to any member of the Schizomycetes class. Bacteria may be round, rodlike, spiral, or filamentous, may be single-celled or noncellular, and may either be motile or aggregated into colonies. Examples of bacteria include, but are not limited to, Clostridium, Vancomycin-resistant Enterococcus, Helicobacter pylori, Campylobacter, Salmonella non-typhoid, enterohemorrhagic E. coli (EHEC), enteropathogenic E. coli (EPEC), enterotoxigenic E. coli (ETEC), enteroaggregative E. coli, Shigella, Vibrio cholerae, Staphylococcus, Streptococcus, Yersinia, Listeria, Bacillus cereus, Bacillus anthracis, Francisella tularensis, Gardnerella vaginalis, L. acidophilus, Bacteroides, Hemophilis vaginalis, Mobiluncus, Mycoplasma hominis, Chlamydia trachomatis, Ureaplasma urealyticum, Neisseria, Hemophilus, Pneumococci, Bordetella, Corynebacterium and Gonorrhea. The term “fungus” refers to any member of the group Fungi, such as molds, rusts, mildew, smuts, mushrooms, and yeasts. Examples of fungi include, but are not limited to, Histoplasmas, Blastomycetes, Coccidioides, Paracoccidioides, Candida, Torulopsis, and Aspergillus. The term “parasite” refers to an animal or plant living in or on an organism of another species (its host), obtaining from it part or all of its organic nutriment, and commonly exhibiting some degree of adaptive structural modification. The host is typically, but not always, harmed by the presence of the parasite; it never benefits from this presence. Examples of parasites include, but are not limited to, Plasmodium, Giardia, Cryptosporidium, Entamoeba, Cylospora cayetanensis, Trichomonas vaginalis and Mycoplasmas. The term “virus” refers to any member of a group of infective agents that typically contain a protein coat surrounding a core of genetic material (either RNA or DNA) and are capable of growth and multiplication only in living cells. Examples of viruses include, but are not limited to include Rotavirus, Norwalk virus, Astrovirus, Hepatitis B, hantaviruses, rabies virus, Human Immunodeficiency Virus, herpes simplex virus, and human papillomavirus.

The term “non-absorbable” refers to the absolute inability, relative inability, or lessened ability of the subject compositions to be taken up by a treated subject's tissues or bloodstream. A subject composition with lessened ability to be taken up by a subject's tissues or bloodstream may, for instance, be slowly absorbable. A subject composition with relative inability to be taken up by a subject's tissues or bloodstream may be absorbed much more slowly than a composition known to be absorbable.

The term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single-stranded (such as sense or antisense) and double-stranded polynucleotides. Exemplary nucleic acids for use in the subject invention include antisense, decoy molecules, recombinant genes (including transgenes) and the like. The term “nucleic acid” encompasses “aptamers”, which are single-stranded nucleic acid molecules that have been developed to bind a molecular target, usually by in vitro selection methods.

The term “orifice” as used herein, refers to any opening in the body of a subject. Non-limiting examples of orifices include the anus, mouth, ear, nostrils, vagina, and urethra. The term “orificial disease” or “orificial infection” refers to the establishment of a microorganism, such as a bacterium, virus, fungus, or parasite anywhere in a host's orifices, as well as the act or process of such establishment, and any resulting state in the host from said establishment, act, or process. Non-limiting examples of orificial diseases include diseases and infections of the mouth and vagina. Non-limiting examples of oral infections include thrush, periodontal disease, stomatitis, and the like. Non-limiting examples of vaginal infections include, vaginitis, yeast infection, herpes infection, and the like. A “microorganism capable of producing a orificial disease or infection” is any microorganism that may establish itself in a host's orificial tract. Non-limiting examples of such microorganisms are described in section 3.6.

The term “partial agonist” is art-recognized and refers to a compound that binds to a receptor site but does not produce the maximal effect regardless of its concentration.

A “patient,” “subject” or “host” to be treated by the subject method may mean either a human or non-human animal.

The term “peptide” or “polypeptide” refers to the class of molecules made up of a single chain of amino acid residues linked by peptide bonds. Peptides yield two or more amino acids on hydrolysis, and may form the constituent parts of a protein. The term as used herein encompasses both peptides that are derived from proteins, as well as those that are synthetically produced. The terms further encompasses peptides with naturally-occurring amino acid sequences, those with designed or randomly synthesized sequences, peptideomimetics, retro peptides, and variants of any one peptide.

The term “peptidomimetic” refers to a molecule containing peptide-like structural elements that is capable of mimicking the biological action (s) of a natural parent polypeptide.

The term “pharmaceutically-acceptable salts” is art-recognized and refers to the relatively non-toxic, inorganic and organic acid addition salts of molecules, including, for example, coordination complexes of the present invention.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any supplement or composition, or component thereof, from one organ, or portion of the body, to another organ, or portion of the body. Each carrier must be “acceptable” in the sense of being compatible with the other ingredients of the supplement and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “particle” refers to a soluble, partially soluble, or insoluble 1 to 100 micron entity to which a chemical moiety can be covalently bonded by reaction with a functional group on the entity. The term “particle” includes beads composed of a polymer, as well as of polymeric macromolecules. Many suitable polymeric materials that could comprise particles are known, and include soluble polymers such as polyethylene glycols or polyvinyl alcohols, insoluble polymers such as polystyrene resins, and controlled-release or biodegradable polymers, such as polyglycolides. The term “particle” also includes other materials onto which chemical moieties may be covalently bonded. A suitable particle includes functional groups such as those described below. A particle is termed “soluble” if a particle is soluble under the conditions employed. However, in general, a soluble particle can be rendered insoluble under defined conditions. Accordingly, a particle may be soluble under certain conditions and insoluble under other conditions.

“Protein”, “polypeptide” and “peptide” may be used interchangeably herein when referring to a naturally occurring or recombinant gene product, e.g., as may be encoded by a coding sequence. A “polypeptide” or “peptide” also may refer to a polymer of amino acids, either naturally occurring or synthetically produced. By “gene product” it is meant a molecule that is produced as a result of transcription of a gene. Gene products include RNA molecules transcribed from a gene, as well as proteins translated from such transcripts.

A “peptide nucleic acid” or “PNA” refers to an analogue of a nucleic acid in which the backbone of the molecule is not sugar-phosphate, but rather a peptide or peptidomimetic. A detailed description of PNAs may be found in Nielsen, et al. Curr. Issues Mol. Biol. (1999) 1:89-104.

The terms “recombinant protein,” “heterologous protein” and “exogenous protein” are art-recognized and are used interchangeably to refer to a polypeptide which is produced by recombinant DNA techniques, wherein generally, DNA encoding the polypeptide is inserted into a suitable expression vector which is in turn used to transform a host cell to produce the heterologous protein. That is, the polypeptide is expressed from a heterologous nucleic acid.

“Small molecule” refers to a composition, which has a molecular weight no more than about 20 kDa. Small molecules may be nucleic acids, peptides, peptide-nucleic acids, aptamers polypeptides, peptidomimetics, or other organic (carbon-containing) or inorganic molecules. As those skilled in the art will appreciate, based on the present description, extensive libraries of chemical and/or biological mixtures, often fungal, bacterial, or algal extracts, may be screened with any of the assays of the invention to identify molecules that bind a microorganism.

A “reversed” or “retro” peptide sequence refers to that part of an overall sequence of covalently-bonded amino acid residues (or analogs or mimetics thereof) wherein the normal carboxyl-to amino direction of peptide bond formation in the amino acid backbone has been reversed such that, reading in the conventional left-to-right direction, the amino portion of the peptide bond precedes (rather than follows) the carbonyl portion. See, generally, Goodman et al. Accounts of Chem. Res. 12:423 (1979).

The reversed orientation peptides described herein include (a) those wherein one or more amino-terminal residues are converted to a reversed (“rev”) orientation (thus yielding a second “carboxyl terminus” at the left-most portion of the molecule), and (b) those wherein one or more carboxyl-terminal residues are converted to a reversed (“rev”) orientation (yielding a second “amino terminus” at the right-most portion of the molecule). A peptide (amide) bond cannot be formed at the interface between a normal orientation residue and a reverse orientation residue.

Therefore, certain reversed peptide molecules of the invention may be formed by utilizing an appropriate amino acid mimetic moiety to link the two adjacent portions of the sequences depicted above utilizing a reversed peptide (reversed amide) bond.

The reversed direction of bonding in such molecules will generally, in addition, require inversion of the enantiomeric configuration of the reversed amino acid residues in order to maintain a spatial orientation of side chains that is similar to that of the non-reversed peptide. The configuration of amino acids in the reversed portion of the peptides is usually (D), and the configuration of the non-reversed portion is usually (L). Opposite or mixed configurations are acceptable when appropriate to optimize a binding activity.

A “target” refers to a site to which the compositions of the present invention bind. A target may be either in vivo or in vitro. In certain embodiments, a target may be a site of infection (e.g., by bacteria, viruses, pathogenic fungi, and parasites. Certain target infectious organisms include those that are drug resistant. In still other embodiments, a target may refer to a molecular structure to which a targeting moiety binds, such as a hapten, epitope, receptor, dsDNA fragment, carbohydrate or enzyme. Additionally, a target may be a type of tissue, e.g. intestinal tissue. “Target cells”, which may serve as the target for the method or compositions of the present invention, include prokaryotes and eukaryotes, including yeasts, plant cells and animal cells. Such cells, when they comprise bacteria, viruses, parasites, or fungi, are referred to as “target microorganisms”.

The term “targeting moiety” refers to any molecular structure which assists the construct in localizing to a particular target area, entering a target cell(s), and/or binding to a target receptor. For example, nucleic acids, antibodies, ligands, steroids, hormones, nutrients, and proteins may serve as targeting moieties.

The term “therapeutic effect” is art-recognized and refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically effective amount of a compound will depend on its therapeutic index, solubility, and the like. For example, certain compositions of the present invention may be administered in a sufficient amount to produce a at a reasonable benefit/risk ratio applicable to such treatment.

The term “therapeutic index” is art-recognized and refers to the therapeutic index of a drug or other compound or particle able to bind a gastrointestinal microorganism defined as LD₅₀/ED₅₀.

The term “treating” is art-recognized and refers to curing as well as ameliorating at least one symptom of any condition or disease.

The term “prophylactic” or “therapeutic treatment” is art-recognized and refers to administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

A “variant” of polypeptide X refers to a polypeptide having the amino acid sequence of peptide X in which is altered in one or more amino acid residues. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological or immunological activity may be found using computer programs well known in the art, for example, LASERGENE software (DNASTAR).

2. Subject Compositions

The present invention was developed as an alternative to traditional antimicrobial compounds. The non-absorbable pharmaceutical compositions of the present invention may bind a variety of surface molecules on gastrointestinal microorganisms and are subsequently cleared from the gastrointestinal tract. The compositions are designed to bind specifically to the target microorganisms, and thus do not remove the normal flora. Such a mode of action is expected to reduce or eliminate the conditions that facilitate the development of microbial resistance. Because the compositions have an extracellular mode of action, they may exhibit few side effects. Furthermore, because multiple receptors may be targeted in the same embodiment of the compositions of the present invention, the composition may be made serotype-independent or may be able to bind more than one species of microorganism.

In certain embodiments, the compositions of the present invention may contain biologically active agents that are released when the composition is ingested. Because the compositions localize to the microorganism they bind, the release of such biologically active agents may stay localized to that area. In other embodiments, the compositions of the present invention may contain agents able to kill a microorganism. Thus, when a composition is localized to a microorganism by binding to it, the microorganism may be brought into contact with said agents.

In one embodiment of the present invention, the non-absorbable compositions are comprised of a particle of the following formula:

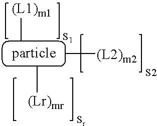

wherein;

L1, L2 . . . Lr, each independently, represent a molecule that is able to bind or kill a gastrointestinal or other microorganism; wherein said molecule is selected from the group consisting of lipids, carbohydrates, peptides, peptidomimetics, peptide-nucleic acids (PNAs), proteins, small molecules, natural products, aptamers and oligonucleotides; and wherein each Lr optionally may be conjugated to said particle through a linker molecule;

mr, independently for each Lr, is at least 1;

sr, independently for each Lr, is at least 1;

and r is at least 2.

In one embodiment of the invention, the non-absorbable compositions are comprised of a particle of the following formula:

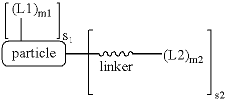

wherein;

L1 and L2 are as defined above; wherein L1 optionally may be conjugated to said particle through a linker molecule of the same type or a different type than the linker through which L2 is conjugated to said particle;

mr, independently for each Lr, is at least 1; and

sr, independently for each Lr, is at least 1.

In one embodiment of the invention, the non-absorbable compositions are comprised of a particle of the following formula:

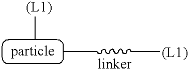

wherein;

L1 is as defined above; wherein some L1 optionally may be conjugated to said particle through a linker molecule of a different type than the linker through which other L1 are conjugated to said particle; and

at least two different L1-particle linkages are present.

In another embodiment of the invention, the non-absorbable compositions are comprised of a particle of the following formula:

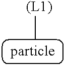

wherein;

L1 is as defined above; wherein L1 optionally may be conjugated to said particle through a linker molecule; and

said particle is able to release biologically active agents, contains agents able to kill a target microorganism, or is otherwise able to aid in the binding or killing of the target microorganism.

In another embodiment of the present invention, the non-absorbable compositions are comprised of a particle of the following formula:

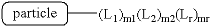

wherein;

L1, L2 . . . Lr, are as previously defined; wherein multiple Lr are conjugated to the particle through a single point of attachment, optionally through a linker molecule, and may be arranged in any order;

r is at least 2; and

mr, independently for each Lr, is at least 1.

The Lr in the above formula may be attached to each other sequentially in any order (at random or in blocks), optionally with linker molecules between them. Alternatively, the Lr may be attached to the conjugating moiety via a branched molecule, as in the following exemplary formula:

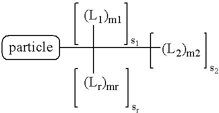

wherein;

L1, L2 . . . Lr, are as previously defined;

each Lr may be linked to the conjugating moiety through the same or different linkages;

r is at least 2;

s2, independently for each Lr, is at least 1; and

mr, independently for each Lr, is at least 1.

By combining different Lr molecules linked to the particles via different chemical moieties, a composition may be able to more effectively bind or kill one or more target microorganisms than could a composition comprising a particle conjugated to a single ligand using identical moieties. Any combination of Lr molecules, conjugation chemistries, and/or linkers are contemplated for use in the present invention. The size, length, and physicochemical (e.g. distribution of functional groups, hydrophobicity, rigidity) properties of the linkers and ligands may be varied and combined onto a single subject particle. Such a variety of different ligands conjugated to a single particle, may, for instance, allow a ligand complex to be formed on the surface of the particle either before or after binding a target microorganism. For example, a complex of protein ligands (e.g. where Lr are proteins) on the surface of a particle could be used to bind a surface molecule of a microorganism.

3. Particles

3.1 Materials from which Particles May be Comprised

The particles of the present invention may be comprised of any material to which the Lr molecules can be conjugated. In certain embodiments, the material for the particle may be chosen so as to effect the optimum binding of the particle to the target microorganisms. For example, negatively or positively charged material may be utilized depending on the charge of the target the composition is being designed to bind. Likewise, a hydrophobic material may be utilized if the target contained significant areas of hydrophobicity. In other embodiments, the material for the particle may contain or be able to release biologically active agents or agents able to kill a target microogranism.

In one embodiment of the invention, the particles are comprised of polymers. Examples of suitable candidate polymers include, but are not limited to, polystyrene, polymethylmethacrylate, polyethylene glycol, polypropylene, polycarbonate, polyethylene, polyurethane, polypropylene glycol, expanded polytetrafluoroethylenes, fluorinated ethylene propylene, polyvinylalcohol, polycarbonate, polylactides, polyglycolids, polycaprolactides, polyarylates, polyanhydrides, and polyphosphoesters.

In certain embodiments, the particle may be a bead. In one embodiment, the bead has a diameter in the range of about 1 to about 50 microns. The bead may in certain embodiments be comprised of any single polymeric molecule, or may be a mix of several polymeric molecules. The charge, lipophilicity or hydrophilicity of any subject particle may be modified by employing such mixtures. Methods by which to prepare beads of controlled size and surface graft density are well-known in the art, and many suitable beads are commercially available from vendors such as DYNAL, Inc, and Sigma-Aldrich-Fluka.

In certain embodiments, the particle may be a macromolecular polymeric compound. Any of the above-mentioned polymer molecules may comprise the macromolecular compound. In certain embodiments, such macromolecular compounds may be comprised entirely of one type of polymeric molecule. In other embodiments, the macromolecular compounds may be comprised of more than one type of polymeric molecule. The charge, lipophilicity or hydrophilicity of any subject particle may be modified by employing such mixtures of molecules. The macromolecular compounds may exist in many possible structures, for example, linear, comb-branched, dendrigraft, dendrimer, or a linear dendron architectural copolymer.

In other embodiments, the particle, either a bead or macromolecule, is comprised of a controlled release polymer. In certain embodiments, the controlled release polymer may contain a biologically active agent. In one embodiment, the controlled release polymer may surround, or encapsulate the agent. In other embodiments, the agent may be evenly distributed throughout the controlled release polymer. A number of biologically active agents are contemplated for use with the present invention, which are described in the following subsection. In certain embodiments, a large percentage of the subject particles may be a biologically active agent. For example, the biologically active agent may comprise 10 to 50% or more of the subject particle, e.g., at least 20%, at least 25%, at least 30%, or more of the composition. The subject compositions may in some embodiments allow high loading levels of a biologically active agent to be incorporated, which allows in certain cases a smaller amount of the subject compositions to be used for treatment with the same therapeutic effect.

Plasticizers and stabilizing agents known in the art may be incorporated in compositions of the present invention. In certain embodiments, additives such as plasticizers and stabilizing agents may be selected for their biocompatibility.

A particle of this invention may further contain one or more adjuvant substances, such as fillers, thickening agents or the like. In other embodiments, materials that serve as adjuvants may be associated with the composition. Such additional materials may affect the characteristics of the composition that results. For example, fillers, such as bovine serum albumin (BSA) or mouse serum albumin (MSA), may be associated with the polymer composition. In certain embodiments, the amount of filler may range from about 0.1 to about 50% or more by weight of the composition, or about 2.5, 5, 10, 25, 40 percent. Incorporation of such fillers may affect the biodegradation of the polymeric material and the sustained release rate of any encapsulated substance. Other fillers known to those of skill in the art, such as carbohydrates, sugars, starches, saccharides, celluloses and polysaccharides, including mannitose and sucrose, may be used in certain embodiments in the present invention.

Buffers, acids and bases may be incorporated in the compositions to adjust their pH. Agents to increase the diffusion distance of agents released from the polymer composition may also be included.

The charge, lipophilicity or hydrophilicity of any subject particle also may be modified by employing an additive. For example, surfactants may be used to enhance miscibility of poorly miscible liquids. Examples of suitable surfactants include dextran, polysorbates, and sodium lauryl sulfate. In general, surfactants are used in low concentrations, generally less than about 5%.

The particles may additionally contain one or more optional additives such as colorants, perfumes, modifying agents, etc. In practice, each of these optional additives should be compatible with the resulting particle and its intended use. The amount of each of these optional additives employed in the composition is an amount necessary to achieve the desired effect.

In certain embodiments, the particles are comprised of germicidal or antimicrobial compounds. Examples of such compounds are quaternary ammonium compounds, silver ions, mercurial compounds, iodine, chlorhexidine, acridine, diamidines, amphipathic polycations such as N-alkylated poly(4-vinylpyridine), quaternized polyurethanes, and the like. (Grapski, J. A., and Cooper, S. L., Biomaterials (2001), 22:2239-46; Tiller, J. C., et al., PNAS (2001),98:5981-5985) Such compounds may comprise the material of the particle itself, e.g. a polymer such as quaternized polyurethanes. In other embodiments, the germicidal or antimicrobial compounds may be incorporated within the material from which the particle, e.g. in a controlled release polymer, or simply mixed in with the material.

3.2 Modification and Derivatization of Particles for Use in the Compositions

The particles of the present invention may be covalently attached to at least one molecule, Lr, that is able to bind or kill a target microorganism. Thus, material comprising the particle of the invention must be modified or derivatized in order to enable the coupling of Lr. For example, to allow coupling of chemicals and linker molecules, the surface chemical groups of a particle can be derivatized with carboxyl (COOH), amino (NH₂), hydroxyl (OH), hydrazide (NHNH₂), amide (CONH₂), chloromethyl (CH₃Cl), and aldehyde (COH) groups. Such strategies for derivatizing and modifying various chemical groups (such as surface hydroxyl or amino groups) to allow coupling of lipids, carbohydrates, peptides, peptidomimetics, peptide-nucleic acids (PNAs), proteins, small molecules, natural products, aptamers and oligonucleotides to a surface are well-known in the art.

For example, polymers can be modified by increasing the number of carboxylic groups accessible during biodegradation, or on the polymer surface. The polymers can also be modified by binding amino groups to the polymer. The polymers can also be modified using any of a number of different coupling chemistries that covalently attach ligand molecules to the surface-exposed molecules of the particles. Certain embodiments of the invention include polymers that have been carboxyl- and amino-modified. Covalent coupling via such moieties creates a high stability linkage between the polymer and molecule.

One useful protocol involves the “activation” of hydroxyl groups on polymer chains with the agent, carbonyldiimidazole (CDI) in aprotic solvents such as DMSO, acetone, or THF. CDI forms an imidazolyl carbamate complex with the hydroxyl group which may be displaced by binding the free amino group of a ligand such as a protein. The reaction is an N-nucleophilic substitution and results in a stable N-alkylcarbamate linkage of the ligand to the polymer. The “coupling” of the ligand to the “activated” polymer matrix is maximal in the pH range of 9-10 and normally requires at least 24 hrs. The resulting ligand-polymer complex is stable and resists hydrolysis for extended periods of time.

Another coupling method involves the use of 1-ethyl-3-(3-dimethylaminopropyl) carbodiimide (EDAC) or “water-soluble CDI” in conjunction with N-hydroxylsulfosuccinimide (sulfo NHS) to couple the exposed carboxylic groups of polymers to the free amino groups of ligands in a totally aqueous environment at the physiological pH of 7.0. Briefly, EDAC and sulfo-NHS form an activated ester with the carboxylic acid groups of the polymer which react with the amine end of a ligand to form a peptide bond. The resulting peptide bond is resistant to hydrolysis. The use of sulfo-NHS in the reaction increases the efficiency of the EDAC coupling by a factor of ten-fold and provides for exceptionally gentle conditions that ensure the viability of the ligand-polymer complex. By using either of these protocols it is possible to “activate” almost all polymers containing either hydroxyl or carboxyl groups in a suitable solvent system that will not dissolve the polymer matrix.

To create an amino-modified polymer, the addition of glutaraldehyde creates an aldehyde-activated surface, which can be attached to a molecule with a free amine. Amino groups on polymers may react with the bis-aldehyde molecule glutaraldehyde to form derivatives able to cross-link with amino-groups. The reaction mechanism for this modification can proceed by different ways. The more simple of these is the formation of a Schiff base linkage between one of the aldehyde ends and amines on the polymer to leave the other aldehyde terminal free to conjugate with another molecule. Schiff base interactions between aldehydes and amines typically are not stable enough to form irreversible linkages, and have to be reduced with suitable reductants. Amino modifying a polymer in this fashion results in a free binding moiety 11-12 carbon atoms away from the polymer, while carbodiimide results in 2-3 carbon linker. Additional linkers may be added to such moieties in order to space the Lr molecule from the particle, to provide flexibility, or to provide a particular orientation, and are discussed below in Section 3.4.

Another useful coupling procedure for attaching ligands with free hydroxyl and carboxyl groups to particles involves the use of the cross-linking agent, divinylsulfone. This method would be useful for attaching sugars or other hydroxylic molecules with bioadhesive properties to hydroxylic matrices. Briefly, the activation involves the reaction of divinylsulfone to the hydroxyl groups of a particle, forming the vinylsulfonyl ethyl ether of the particle. The vinyl groups will couple to alcohols, phenols and even amines. Activation and coupling take place at pH 11. The linkage is stable in the pH range from 1-8 and is suitable for transit through the intestine.

Any suitable coupling method known to those skilled in the art for the coupling of ligands and polymers with double bonds, including the use of UV crosslinking, may be used for attachment of molecules to the particles described herein.

3.3. Biologically Active Agents for Encapsulation or Incorporation in the Particles

In certain embodiments, the compositions of the present invention include both (a) a biologically active agent, and (b) a particle that binds a gastrointestinal microorganism comprised of a material that is able to release said agent. In other embodiments, the particle is comprised in part by the biologically active agent. In certain embodiments the agent comprises an Lr molecule of the invention, and is able to be released from the particle by decomposition of its conjugating moiety. For example, the subject compositions may contain a ‘drug’, ‘therapeutic agent’, ‘medicament’, ‘biologically active agent’, or ‘bioactive substance’, which are biologically, physiologically, or pharmacologically active substances that act locally or systemically in the human or animal body. Various forms of the medicaments or biologically active materials may be used which are capable of being released from the polymer composition. They may be acidic, basic, or salts. They may be neutral molecules, polar molecules, or molecular complexes capable of hydrogen bonding. They may be in the form of ethers, esters, amides and the like, which are biologically activated when injected into the subject.

A particle may, release such agents in addition to its main binding and clearing activity for a variety of reasons. Non-limiting examples of such reasons include: 1) supplementing the treatment of the infection (e.g. release antibiotic locally in addition to binding and clearing the microbes); 2) ameliorating a symptom of the infection (e.g. pain, nausea, inflammation); and 3) enhancing the immune response at the site of infection.

Non-limiting examples of biologically active substances include the following expanded therapeutic categories: androgenic steroids, antacids, anti-diarrheals, anti-emetics, anti-infective agents, anti-inflammatory agents such as steroids, non-steroidal anti-inflammatory agents, anti-malarials, anti-nauseants, anti-pyretic and analgesic agents, anti-spasmodic agents, appetite suppressants, benzophenanthridine alkaloids, biologicals, diuretics, diagnostic agents, gastrointestinal sedatives, humoral agents, ion exchange resins, laxatives, mineral supplements, nutritional substances, sedatives, stimulants, tranquilizers, vitamins, antigenic materials, and prodrugs.

Specific examples of useful biologically active substances include: (a) ion exchange resins such as cholestyramine; (b) antipyretics and analgesics such as acetaminophen, aspirin and ibuprofen; (c) biologicals such as peptides, polypeptides, proteins and amino acids, hormones, interferons or cytokines and other bioactive peptidic compounds, such as hGH, tPA, calcitonin, ANF, EPO and insulin; (d) anti-infective agents such as anti-fungals, anti-virals, antiseptics and antibiotics; and (e) desensitizing agents and antigenic materials, such as those useful for vaccine applications.

More specifically, non-limiting examples of useful biologically active substances include the following therapeutic categories: analgesics, such as nonsteroidal anti-inflammatory drugs, opiate agonists and salicylates; anti-infective agents, such as antihelmintics, antianaerobics, antibiotics, aminoglycoside antibiotics, antifungal antibiotics, cephalosporin antibiotics, macrolide antibiotics, miscellaneous β-lactam antibiotics, penicillin antibiotics, quinolone antibiotics, sulfonamide antibiotics, tetracycline antibiotics, antimycobacterials, antituberculosis antimycobacterials, antiprotozoals, antimalarial antiprotozoals, antiviral agents, anti-retroviral agents, scabicides, anti-inflammatory agents, corticosteroid anti-inflammatory agents, antipruritics/local anesthetics, topical anti-infectives, antifungal topical anti-infectives, antiviral topical anti-infectives; electrolytic and renal agents, such as acidifying agents, alkalinizing agents, diuretics, carbonic anhydrase inhibitor diuretics, loop diuretics, osmotic diuretics, potassium-sparing diuretics, thiazide diuretics, electrolyte replacements, and uricosuric agents; enzymes, such as pancreatic enzymes and thrombolytic enzymes; gastrointestinal agents, such as antidiarrheals, antiemetics, gastrointestinal anti-inflammatory agents, salicylate gastrointestinal anti-inflammatory agents, antacid anti-ulcer agents, gastric acid-pump inhibitor anti-ulcer agents, gastric mucosal anti-ulcer agents, H₂-blocker anti-ulcer agents, cholelitholytic agents, digestants, emetics, laxatives and stool softeners, and prokinetic agents; general anesthetics, such as inhalation anesthetics, halogenated inhalation anesthetics, intravenous anesthetics, barbiturate intravenous anesthetics, benzodiazepine intravenous anesthetics, and opiate agonist intravenous anesthetics; hormones and hormone modifiers, such as abortifacients, adrenal agents, corticosteroid adrenal agents, androgens, anti-androgens, immunobiologic agents, such as immunoglobulins, immunosuppressives, toxoids, and vaccines; local anesthetics, such as amide local anesthetics and ester local anesthetics; musculoskeletal agents, such as anti-gout anti-inflammatory agents, corticosteroid anti-inflammatory agents, gold compound anti-inflammatory agents, immunosuppressive anti-inflammatory agents, nonsteroidal anti-inflammatory drugs (NSAIDs), salicylate anti-inflammatory agents, minerals; and vitamins, such as vitamin A, vitamin B, vitamin C, vitamin D, vitamin E, and vitamin K.

Preferred classes of useful biologically active substances from the above categories include: (1) analgesics in general, such as lidocaine or derivatives thereof, and nonsteroidal antiinflammatory drugs (NSAIDs) analgesics, including diclofenac, ibuprofen, ketoprofen, and naproxen; (2) opiate agonist analgesics, such as codeine, fentanyl, hydromorphone, and morphine; (3) salicylate analgesics, such as aspirin (ASA) (enteric coated ASA); (4) H₁-blocker antihistamines, such as clemastine and terfenadine; (5) anti-infective agents, such as mupirocin; (6) antianaerobic anti-infectives, such as chloramphenicol and clindamycin; (7) antifungal antibiotic anti-infectives, such as amphotericin b, clotrimazole, fluconazole, and ketoconazole; (8) macrolide antibiotic anti-infectives, such as azithromycin and erythromycin; (9) miscellaneous 13-lactam antibiotic anti-infectives, such as aztreonam and imipenem; (10) penicillin antibiotic anti-infectives, such as nafcillin, oxacillin, penicillin G, and penicillin V; (11) quinolone antibiotic anti-infectives, such as ciprofloxacin and norfloxacin; (12) tetracycline antibiotic anti-infectives, such as doxycycline, minocycline, and tetracycline; (13) antituberculosis antimycobacterial anti-infectives such as isoniazid (INH), and rifampin; (14) antiprotozoal anti-infectives, such as atovaquone and dapsone; (15) antimalarial antiprotozoal anti-infectives, such as chloroquine and pyrimethamine; (16) anti-retroviral anti-infectives, such as ritonavir and zidovudine; (17) antiviral anti-infective agents, such as acyclovir, ganciclovir, interferon alfa, and rimantadine; (18) antifungal topical anti-infectives, such as amphotericin B, clotrimazole, miconazole, and nystatin; (19) antiviral topical anti-infectives, such as acyclovir; (20) electrolytic and renal agents, such as lactulose; (21) loop diuretics, such as furosemide; (22) potassium-sparing diuretics, such as triamterene; (23) thiazide diuretics, such as hydrochlorothiazide (HCTZ); (24) unricosuric agents, such as probenecid; (25) enzymes such as RNase and DNase; (26) antiemetics, such as prochlorperazine; (27) salicylate gastrointestinal anti-inflammatory agents, such as sulfasalazine; (28) gastric acid-pump inhibitor anti-ulcer agents, such as omeprazole; (29) H₂-blocker anti-ulcer agents, such as cimetidine, famotidine, nizatidine, and ranitidine; (30) digestants, such as pancrelipase; (31) prokinetic agents, such as erythromycin; (32) ester local anesthetics, such as benzocaine and procaine; (33) musculoskeletal corticosteroid anti-inflammatory agents, such as beclomethasone, betamethasone, cortisone, dexamethasone, hydrocortisone, and prednisone; (34) musculoskeletal anti-inflammatory immunosuppressives, such as azathioprine, cyclophosphamide, and methotrexate; (35) musculoskeletal nonsteroidal anti-inflammatory drugs (NSAIDs), such as diclofenac, ibuprofen, ketoprofen, ketorlac, and naproxen; (36) minerals, such as iron, calcium, and magnesium; (37) vitamin B compounds, such as cyanocobalamin (vitamin B₁₂) and niacin (vitamin B₃); (38) vitamin C compounds, such as ascorbic acid; and (39) vitamin D compounds, such as-calcitriol.

Further, recombinant or cell-derived proteins such as recombinant beta-glucan; bovine immunoglobulin concentrate; bovine superoxide dismutase; recombinant interferon beta-1a; and lenograstim (G-CSF) may be used.

Still further, the following listing of peptides, proteins, and other large molecules may also be used, such as interleukins 1 through 18, including mutants and analogues; interferons α, γ, and β; luteinizing hormone releasing hormone (LHRH) and analogues, transforming growth factor-α (TGF-α); fibroblast growth factor (FGF); invasion inhibiting factor-2 (IIF-2); thymosin-α-1; γ-globulin; superoxide dismutase (SOD); and complement factors.

In certain embodiments, the biologically active substance is selected from the group consisting of polysaccharides, growth factors, hormones, anti-angiogenesis factors, interferons or cytokines, antigenic materials, and pro-drugs. In one embodiment, the biologically active substance is a therapeutic drug or pro-drug, most preferably a drug selected from the group consisting of antibiotics, anti-virals, anti-fungals, anti-inflammatories, and antigens useful for vaccine applications or corresponding pro-drugs.

In still other embodiments, the biologically active agent may be a non-antibiotic bacteriocidal molecule or compound. Examples of these are quaternary ammonium compounds, silver ions, mercurial compounds, iodine, chlorhexidine, acridine, diamidines, amphipathic polycations such as N-alkylated poly(4-vinylpyridine), quaternized polyurethanes, and the like.

In certain embodiments, the biologically active agent may be a chemoattractant. A compositions comprised of a particle containing such an agent may “lure” the target microbe to it, thus aiding in the binding and clearance of the microbes by the composition. Non-limiting examples of chemoattractants that may be releases by a subject particle include carbohydrates, amino acids, nitrates, protons, and salts.

4. Linkers

Linkers (also known as “linker molecules” or “cross-linkers” or “spacers”) may be used to conjugate particles and a subject Lr molecule. In certain embodiments, the linker acts to simultaneously derivatize and conjugate an Lr molecule to the molecule. In other embodiments, an additional linker is used to conjugate a derivatized particle and an Lr molecule. Linkers are chemicals able to react with a defined chemical group of several, usually two, molecules and thus conjugate them. The majority of known cross-linkers react with amine, carboxyl, and sulfhydryl groups. The choice of target chemical group is crucial if the group may be involved in the biological activity of the molecule Lr to be conjugated to the particle. For example, maleimides, which react with sulfhydryl groups, may inactivate Cys-containing peptides or proteins that require the Cys to bind a target. Linkers may be homofunctional (containing reactive groups of the same type), heterofunctional (containing different reactive groups), or photoreactive (containing groups that become reactive on illumination).

Linker molecules may be responsible for different properties of the conjugated composition. The length of the linker should be considered in light of molecular flexibility during the conjugation step, and the availability of the conjugated molecule for its target (cell surface molecules and the like.) Longer linkers may thus improve the biological activity of the compositions of the present invention, as well as the ease of preparation of them. The geometry of the linker may be used to orient a molecule for optimal reaction with a target. A linker with flexible geometry may allow the entire linker-Lr molecule complex to conformationally adapt as it binds a target microorganism. The nature of the linker may be altered for other various purposes. For example, the aryl-structure of MBuS was found less immunogenic than the aromatic spacer of MBS. Furthermore, the hydrophobicity and functionality of the linker molecules may be controlled by the physical properties of component molecules. For example, the hydrophobicity of a polymeric linker may be controlled by the order of monomeric units along the polymer, e.g. a block polymer in which there is a block of hydrophobic monomers interspersed with a block of hydrophilic monomers.

The chemistry of preparing and utilizing a wide variety of molecular linkers is well-known in the art and many pre-made linkers for use in conjugating molecules are commercially available from vendors such as Pierce Chemical Co., Roche Molecular Biochemicals, United States Biological

Exemplary linker molecules for use in the compositions of the present invention include, but are not limited to: aminocaproic acid (ACA); polyglycine, and any other amino acid polymer, polymers such as polyethylene glycol (PEG), polymethyl methacrylate (PMMA), polypropylene glycol (PPG); homobifunctional reagents such as APG, AEDP, BASED, BMB, BMDB, BMH, BMOE, BM[PEO]3, BM[PEO]4, BS3, BSOCOES, DFDNB, DMA, DMP, DMS, DPDPB, DSG, DSP (Lomant's Reagent), DSS, DST, DTBP, DTME, DTSSP, EGS, HBVS, Sulfo-BSOCOES, Sulfo-DST, Sulfo-EGS; heterobifunctional reagents such as ABH, AEDP, AMAS, ANB-NOS, APDP, ASBA, BMPA, BMPH, BMPS, EDC, EMCA, EMCH, EMCS, KMUA, KMUH, GMBS, LC-SMCC, LC-SPDP, MBS, MBuS, M2C2H, MPBH, MSA, NHS-ASA, PDPH, PMPI, SADP, SAED. SAND, SANPAH, SASD, SATP, SBAP, SFAD, SIA, SIAB, SMCC, SMPB, SMPH, SMPT, SPDP, Sulfo-EMCS, Sulfo-GMBS, Sulfo-HSAB, Sulfo-KMUS, Sulfo-LC-SPDP, Sulfo-MBS. Sulfo-NHS-LC-ASA, Sulfo-SADP, Sulfo-SANPAH, Sulfo-SIAB, Sulfo-SMCC, Sulfo-SMPB, Sulfo-LC-SMPT, SVSB, TFCS; and trifunctional linkers such as Sulfo-SBED, THPP B[Tris(hydroxymethyl) phosphino] propionic acid (betaine) Any linkers contemplated for use with the screening methods described in section 3.5 include, but are not limited to, any molecule that does not contain a functionality incompatible with the reaction scheme require to prepare the library members or with the coupling of an Lr molecule.

Branched linkers may be prepared or used so that multiple moieties per linker are able to react with molecule Lr. For example, a branched polymeric linker may have Lr at all of its ends. Such multiply reactive linkers allow the creation of multimeric binding sites, which may enhance the ability of a single linker to bind the target microbe.

In certain embodiments of the present invention, the linker may be a macromolecular polymer. Any of the above-mentioned polymers may comprise the macromolecular polymer. In certain embodiments, such macromolecular polymers may be comprised entirely of one type of polymeric molecule. In other embodiments, the macromolecular polymers may be comprised of more than one type of polymeric molecule. The macromolecular polymers may exist in many possible structures, for example, linear, comb-branched, dendrigraft, dendrimer, or a linear dendron architectural copolymer. For example, PEG and PPG may be used to create a variety of bi- and multivalent linkers. Methods of synthesizing, activating, and modifying branched PEG/PPG polymers and PEG/PPG block co-polymers are well-known in the art. PEG is hydrophilic, while PPG is hydrophobic. For instance, a linker could be synthesized with a PPG core and PEG branches. Such a linker may help the composition interact with a target bacterium's membrane via the linker's PPG core.

5. Lr Molecules that Bind to Microorganisms

As described above, the compositions of the invention comprise particles that are covalently bound to at least one Lr molecule that is able to bind a gastrointestinal or other microorganism. Lr may be selected from the group consisting of lipids, carbohydrates, peptides, peptidomimetics, peptide-nucleic acids (PNAs), proteins, small molecules, natural products, aptamers and oligonucleotides. Each Lr molecule of a particle may be conjugated to the particle by any one of a variety of linker molecules, as described above. Because multiple reactive groups are present on a derivatized particle, multiple Lr-linker molecule moieties will cover the surface of a particle. In certain embodiments, wherein the particle is a polymeric bead, at least 10⁴ Lr-linker moieties may be present. In certain embodiments of the present invention, different Lr, and optionally different linkers for each, may be conjugated to the surface of a particle, enabling one particle to bind a variety of different targets. For example, different Lr directed to targets specific to different serotypes of a microbe may be incorporated onto a single particle. In embodiments wherein the linker molecule is branched, an Lr molecule may be present at the end of each branch, thus creating multiple binding sites per linker.

In certain embodiments, Lr is selected from a library of molecules, which optionally may be prepared through combinatorial synthesis. Lr may be selected from said library by screening its ability to bind a target microoorganism. The methods of the present invention may be in vitro and comprise the use of a molecule on the surface of the target microorganism or the target microorganism itself. The methods may be in vivo and comprise the use of an entire particle bound to a candidate Lr molecule.

In another embodiment of the invention, Lr is able to kill the target microbe. In these embodiments, Lr may be selected from the group consisting of amphipathic polycations, quaternary ammonium compounds (acridine, diamidine), and quaternary polymers such as quaternized polyurethane. (Grapski, J. A., and Cooper, S. L., Biomaterials (2001), 22:2239-46; Tiller, J. C., et al., PNAS (2001),98:5981-5985)

5.1. Chemical Libraries of Candidate Lr Molecules

The synthesis and screening of combinatorial libraries is a validated strategy for the identification and study of organic molecules of interest. According to the present invention, the synthesis of libraries containing molecules that bind or kill a gastrointestinal microorganism may be performed using established combinatorial methods for solution phase, solid phase, or a combination of solution phase and solid phase synthesis techniques. The synthesis of combinatorial libraries is well known in the art and has been reviewed (see, e.g., “Combinatorial Chemistry”, Chemical and Engineering News, Feb. 24, 1997, p. 43; Thompson et al., Chem. Rev. (1996) 96:555). Many libraries are commercially available. One of ordinary skill in the art will realize that the choice of method for any particular embodiment will depend upon the specific number of molecules to be synthesized, the specific reaction chemistry, and the availability of specific instrumentation, such as robotic instrumentation for the preparation and analysis of the inventive libraries. In certain embodiments, the reactions to be performed to generate the libraries are selected for their ability to proceed in high yield, and in a stereoselective and regioselective fashion, if applicable.

In one aspect of the present invention, the inventive libraries are generated using a solution phase technique. Traditional advantages of solution phase techniques for the synthesis of combinatorial libraries include the availability of a much wider range of reactions, and the relative ease with which products may be characterized, and ready identification of library members, as discussed below. For example, in certain embodiments, for the generation of a solution phase combinatorial library, a parallel synthesis technique is utilized, in which all of the products are assembled separately in their own reaction vessels. In a particular parallel synthesis procedure, a microtitre plate containing n rows and m columns of tiny wells which are capable of holding a few milliliters of the solvent in which the reaction will occur, is utilized. It is possible to then use n variants of reactant A, such as a ligand, and m variants of reactant B, such as a second ligand, to obtain n×m variants, in n×m wells. One of ordinary skill in the art will realize that this particular procedure is most useful when smaller libraries are desired, and the specific wells may provide a ready means to identify the library members in a particular well.

In other embodiments of the present invention, a solid phase synthesis technique is utilized. Solid phase techniques allow reactions to be driven to completion because excess reagents may be utilized and the unreacted reagent washed away. Solid phase synthesis also allows the use a technique called “split and pool”, in addition to the parallel synthesis technique, developed by Furka. See, e.g., Furka et al., Abstr. 4th Int. Congr. Biochem., (Prague, Czechoslovakia) (1988) 5:47; Furka et al., Int. J. Pept. Protein Res. (1991) 37:487; Sebestyen et al., Bioorg. Med. Chem. Lett. (1993) 3:413. In this technique, a mixture of related molecules may be made in the same reaction vessel, thus substantially reducing the number of containers required for the synthesis of very large libraries, such as those containing as many as or more than one million library members. As an example, the solid support with the starting material attached may be divided into n vessels, where n represents the number species of reagent A to be reacted with the such starting material. After reaction, the contents from n vessels are combined and then split into m vessels, where m represents the number of species of reagent B to be reacted with the now modified starting materials. This procedure is repeated until the desired number of reagents is reacted with the starting materials to yield the inventive library.

The use of solid phase techniques in the present invention may also include the use of a specific encoding technique. Specific encoding techniques have been reviewed by Czarnik in Current Opinion in Chemical Biology (1997) 1:60. One of ordinary skill in the art will also realize that if smaller solid phase libraries are generated in specific reaction wells, such as 96 well plates, or on plastic pins, the reaction history of these library members may also be identified by their spatial coordinates in the particular plate, and thus are spatially encoded. In other embodiments, an encoding technique involves the use of a particular “identifying agent” attached to the solid support, which enables the determination of the structure of a specific library member without reference to its spatial coordinates. Examples of such encoding techniques include, but are not limited to, spatial encoding techniques, graphical encoding techniques, including the “tea bag” method, chemical encoding methods, and spectrophotometric encoding methods. One of ordinary skill in the art will realize that the particular encoding method to be used in the present invention must be selected based upon the number of library members desired, and the reaction chemistry employed.

In certain embodiments, molecules of the present invention may be prepared using solid support chemistry known in the art. For example, polypeptides having up to twenty amino acids or more may be generated using standard solid phase technology on commercially available equipment (such as Advanced Chemtech multiple organic synthesizers). In certain embodiments, a starting material or later reactant may be attached to the solid phase, through a linking unit, or directly, and subsequently used in the synthesis of desired molecules. The choice of linkage will depend upon the reactivity of the molecules and the solid support units and the stability of these linkages. Direct attachment to the solid support via a linker molecule may be useful if it is desired not to detach the library member from the solid support. For example, for direct on-bead analysis of biological activity, a stronger interaction between the library member and the solid support may be desirable. Alternatively, the use of a linking reagent may be useful if more facile cleavage of the inventive library members from the solid support is desired.

In certain embodiments of the invention, the solid support is the particle on which the molecule Lr will remain to ultimately comprise the compositions able to bind a gastrointestinal microorganism of the present invention. In certain embodiments, a linker molecule is attached to the support before synthesis of the molecule. In other embodiments, both the linker and molecule are combinatorially synthesized onto the support.

In regard to automation of the present subject methods, a variety of instrumentation may be used to allow for the facile and efficient preparation of chemical libraries of the present invention, and methods of assaying members of such libraries. In general, automation, as used in reference to the synthesis and preparation of the subject chemical libraries, involves having instrumentation complete one or more of the operative steps that must be repeated a multitude of times because a library instead of a single molecule is being prepared. Examples of automation include, without limitation, having instrumentation complete the addition of reagents, the mixing and reaction of them, filtering of reaction mixtures, washing of solids with solvents, removal and addition of solvents, and the like. Automation may be applied to any steps in a reaction scheme, including those to prepare, purify and assay molecules for use in the compositions of the present invention.

There is a range of automation possible. For example, the synthesis of the subject libraries may be wholly automated or only partially automated. If wholly automated, the subject library may be prepared by the instrumentation without any human intervention after initiating the synthetic process, other than refilling reagent bottles or monitoring or programming the instrumentation as necessary. Although synthesis of a subject library may be wholly automated, it may be necessary for there to be human intervention for purification, identification, or the like of the library members.

In contrast, partial automation of the synthesis of a subject library involves some robotic assistance with the physical steps of the reaction schema that gives rise to the library, such as mixing, stirring, filtering and the like, but still requires some human intervention other than just refilling reagent bottles or monitoring or programming the instrumentation. This type of robotic automation is distinguished from assistance provided by convention organic synthetic and biological techniques because in partial automation, instrumentation still completes one or more of the steps of any schema that is required to be completed a multitude of times because a library of molecules is being prepared.

In certain embodiments, the subject library may be prepared in multiple reaction vessels (e.g., microtitre plates and the like), and the identity of particular members of the library may be determined by the location of each vessel. In other embodiments, the subject library may be synthesized in solution, and by the use of deconvolution techniques, the identity of particular members may be determined.

In one aspect of the invention, the subject screening method may be carried out utilizing immobilized libraries. In certain embodiments, the immobilized library will have the ability to bind to a microorganism as described above. The choice of a suitable support will be routine to the skilled artisan. Important criteria may include that the reactivity of the support not interfere with the reactions required to prepare the library. Insoluble polymeric supports include functionalized polymers based on polystyrene, polystyrene/divinylbenzene copolymers, and the like, including any of the particles described in section 4.3. It will be understood that the polymeric support may be coated, grafted or otherwise bonded to other solid supports.

In another embodiment, the polymeric support may be provided by reversibly soluble polymers. Such polymeric supports include functionalized polymers based on polyvinyl alcohol or polyethylene glycol (PEG). A soluble support may be made insoluble (e.g., may be made to precipitate) by addition of a suitable inert nonsolvent. One advantage of reactions performed using soluble polymeric supports is that reactions in solution may be more rapid, higher yielding, and more complete than reactions that are performed on insoluble polymeric supports.

Once the synthesis of either a desired solution phase or solid support bound template has been completed, the template is then available for further reaction to yield the desired solution phase or solid support bound structure. The use of solid support bound templates enables the use of more rapid split and pool techniques.

Characterization of the library members may be performed using standard analytical techniques, such as mass spectrometry, Nuclear Magnetic Resonance Spectroscopy, including ¹⁹⁵Pt and ¹H NMR, chromatography (e.g, liquid etc.) and infra-red spectroscopy. One of ordinary skill in the art will realize that the selection of a particular analytical technique will depend upon whether the inventive library members are in the solution phase or on the solid phase. In addition to such characterization, the library member may be synthesized separately to allow for more ready identification.

Detailed descriptions of a number of combinatorial methodologies are provided below.

A) Direct Characterization

A growing trend in the field of combinatorial chemistry is to exploit the sensitivity of techniques such as mass spectrometry (MS), e.g., which can be used to characterize sub-femtomolar amounts of a molecule, and to directly determine the chemical constitution of a molecule selected from a combinatorial library. For instance, where the library is provided on an insoluble support matrix, discrete populations of molecules can be first released from the support and characterized by MS. In other embodiments, as part of the MS sample preparation technique, such MS techniques as MALDI can be used to release a molecule from the matrix, particularly where a labile bond is used originally to tether the molecule to the matrix. For instance, a bead selected from a library can be irradiated in a MALDI step in order to release the diversomer from the matrix, and ionize the diversomer for MS analysis.

B) Multipin Synthesis

The libraries of the subject method can take the multipin library format. Briefly, Geysen and co-workers (Geysen et al. PNAS (1984) 81:3998-4002) introduced a method for generating molecule libraries by a parallel synthesis on polyacrylic acid-grated polyethylene pins arrayed in the microtitre plate format. The Geysen technique can be used to synthesize and screen thousands of molecules per week using the multipin method, and the tethered molecules may be reused in many assays. Appropriate linker moieties can also been appended to the pins so that the molecules may be cleaved from the supports after synthesis for assessment of purity and further evaluation (c.f., Bray et al. Tetrahedron Lett (1990) 31:5811-5814; Valerio et al. Anal Biochem (1991) 197:168-177; Bray et al. Tetrahedron Lett (1991) 32:6163-6166). In certain embodiments of the present invention, the linker and molecule may be coupled onto a particle after evaluation.

C) Divide-Couple-Recombine

In yet another embodiment, a variegated library of molecules can be provided on a set of beads utilizing the strategy of divide-couple-recombine (see, e.g., Houghten PNAS (1985) 82:5131-5135; and U.S. Pat. Nos. 4,631,211; 5,440,016; 5,480,971). Briefly, as the name implies, at each synthesis step where degeneracy is introduced into the library, the beads are divided into separate groups equal to the number of different substituents to be added at a particular position in the library, the different substituents coupled in separate reactions, and the beads recombined into one pool for the next iteration.

In one embodiment, the divide-couple-recombine strategy can be carried out using an analogous approach to the so-called “tea bag” method first developed by Houghten, where molecule synthesis occurs on resin sealed inside porous polypropylene bags (Houghten et al. PNAS (1986) 82:5131-5135). Substituents are coupled to the molecule-bearing resins by placing the bags in appropriate reaction solutions, while all common steps such as resin washing and deprotection are performed simultaneously in one reaction vessel. At the end of the synthesis, each bag contains a single molecule.

D) Combinatorial Libraries by Light-Directed, Spatially Addressable Parallel Chemical Synthesis

A scheme of combinatorial synthesis in which the identity of a molecule is given by its locations on a synthesis substrate is termed a spatially-addressable synthesis. In one embodiment, the combinatorial process is carried out by controlling the addition of a chemical reagent to specific locations on a solid support (Dower et al. Annu Rep Med Chem (1991) 26:271-280; Fodor, S. P. A. Science (1991) 251:767; Pirrung et al. (1992) U.S. Pat. No. 5,143,854; Jacobs et al. (1994) Trends Biotechnol 12:19-26). The spatial resolution of photolithography affords miniaturization. This technique can be carried out through the use protection/deprotection reactions with photolabile protecting groups.

The key points of this technology are illustrated in Gallop et al. J Med Chem (1994) 37:1233-1251. A synthesis substrate is prepared for coupling through the covalent attachment of photolabile nitroveratryloxycarbonyl (NVOC) protected amino linkers or other photolabile linkers. Light is used to selectively activate a specified region of the synthesis support for coupling. Removal of the photolabile protecting groups by light (deprotection) results in activation of selected areas. After activation, the first of a set of amino acid analogs, each bearing a photolabile protecting group on the amino terminus, is exposed to the entire surface. Coupling only occurs in regions that were addressed by light in the preceding step. The reaction is stopped, the plates washed, and the substrate is again illuminated through a second mask, activating a different region for reaction with a second protected building block. The pattern of masks and the sequence of reactants define the products and their locations. Since this process utilizes photolithography techniques, the number of molecules that can be synthesized is limited only by the number of synthesis sites that can be addressed with appropriate resolution. The position of each molecule is precisely known; hence, its interactions with other molecules can be directly assessed.

In a light-directed chemical synthesis, the products depend on the pattern of illumination and on the order of addition of reactants. By varying the lithographic patterns, many different sets of test molecules can be synthesized simultaneously; this characteristic leads to the generation of many different masking strategies.

E) Encoded Combinatorial Libraries

In yet another embodiment, the subject method utilizes a molecule library provided with an encoded tagging system. A recent improvement in the identification of active molecules from combinatorial libraries employs chemical indexing systems using tags that uniquely encode the reaction steps a given bead has undergone and, by inference, the structure it carries. Conceptually, this approach mimics phage display libraries, where activity derives from expressed peptides, but the structures of the active peptides are deduced from the corresponding genomic DNA sequence. The first encoding of synthetic combinatorial libraries employed DNA as the code. A variety of other forms of encoding have been reported, including encoding with sequenceable bio-oligomers (e.g., oligonucleotides and peptides), and binary encoding with additional non-sequenceable tags.

1) Tagging with Sequenceable Bio-Oligomers

The principle of using oligonucleotides to encode combinatorial synthetic libraries was described in 1992 (Brenner et al. PNAS (1992) 89:5381-5383), and an example of such a library appeared the following year (Needles et al. PNAS (1993) 90:10700-10704). A combinatorial library of nominally 7⁷ (=823,543) peptides composed of all combinations of Arg, Gln, Phe, Lys, Val, D-Val and Thr (three-letter amino acid code), each of which was encoded by a specific dinucleotide (TA, TC, CT, AT, TT, CA and AC, respectively), was prepared by a series of alternating rounds of peptide and oligonucleotide synthesis on solid support. In this work, the amine linking functionality on the bead was specifically differentiated toward peptide or oligonucleotide synthesis by simultaneously preincubating the beads with reagents that generate protected OH groups for oligonucleotide synthesis and protected NH₂ groups for peptide synthesis (here, in a ratio of 1:20). When complete, the tags each consisted of 69-mers, 14 units of which carried the code. The bead-bound library was incubated with a fluorescently labeled antibody, and beads containing bound antibody that fluoresced strongly were harvested by fluorescence-activated cell sorting (FACS). The DNA tags were amplified by PCR and sequenced, and the predicted peptides were synthesized. Following such techniques, molecule libraries can be derived for use in the subject method, where the oligonucleotide sequence of the tag identifies the sequential combinatorial reactions that a particular bead underwent, and therefore provides the identity of the molecule on the bead.

The use of oligonucleotide tags permits exquisitely sensitive tag analysis. Even so, the method requires careful choice of orthogonal sets of protecting groups required for alternating co-synthesis of the tag and the library member. Furthermore, the chemical lability of the tag, particularly the phosphate and sugar anomeric linkages, may limit the choice of reagents and conditions that can be employed for the synthesis of non-oligomeric libraries. In preferred embodiments, the libraries employ linkers permitting selective detachment of the test molecule library member for assay.

Peptides have also been employed as tagging molecules for combinatorial libraries. Two exemplary approaches are described in the art, both of which employ branched linkers to solid phase upon which coding and ligand strands are alternately elaborated. In the first approach (Kerr J M et al. J Am Chem Soc (1993) 115:2529-2531), orthogonality in synthesis is achieved by employing acid-labile protection for the coding strand and base-labile protection for the molecule strand.

In an alternative approach (Nikolaiev et al. Pept Res (1993) 6:161-170), branched linkers are employed so that the coding unit and the test molecule can both be attached to the same functional group on the resin. In one embodiment, a cleavable linker can be placed between the branch point and the bead so that cleavage releases a molecule containing both code and the molecule (Ptek et al. Tetrahedron Lett (1991) 32:3891-3894). In another embodiment, the cleavable linker can be placed so that the test molecule can be selectively separated from the bead, leaving the code behind. This last construct is particularly valuable because it permits screening of the test molecule without potential interference of the coding groups. Examples in the art of independent cleavage and sequencing of peptide library members and their corresponding tags has confirmed that the tags can accurately predict the peptide structure.

2) Non-Sequenceable Tagging: Binary Encoding

An alternative form of encoding the test molecule library employs a set of non-sequencable electrophoric tagging molecules that are used as a binary code (Ohlmeyer et al. PNAS (1993) 90:10922-10926). Exemplary tags are haloaromatic alkyl ethers that are detectable as their trimethylsilyl ethers at less than femtomolar levels by electron capture gas chromatography (ECGC). Variations in the length of the alkyl chain, as well as the nature and position of the aromatic halide substituents, permit the synthesis of at least 40 such tags, which in principle can encode 2⁴⁰ (e.g., upwards of 10¹²) different molecules. In the original report (Ohlmeyer et al., supra) the tags were bound to about 1% of the available amine groups of a peptide library via a photocleavable o-nitrobenzyl linker. This approach is convenient when preparing combinatorial libraries of peptide-like or other amine-containing molecules. A more versatile system has, however, been developed that permits encoding of essentially any combinatorial library. Here, the molecule would be attached to the solid support via the photocleavable linker and the tag is attached through a catechol ether linker via carbene insertion into the bead matrix (Nestler et al. J Org Chem (1994) 59:4723-4724). This orthogonal attachment strategy permits the selective detachment of library members for assay in solution and subsequent decoding by ECGC after oxidative detachment of the tag sets.

Although several amide-linked libraries in the art employ binary encoding with the electrophoric tags attached to amine groups, attaching these tags directly to the bead matrix provides far greater versatility in the structures that can be prepared in encoded combinatorial libraries. Attached in this way, the tags and their linker are nearly as unreactive as the bead matrix itself. Two binary-encoded combinatorial libraries have been reported where the electrophoric tags are attached directly to the solid phase (Ohlmeyer et al. PNAS (1995) 92:6027-6031) and provide guidance for generating the subject molecule library. Both libraries were constructed using an orthogonal attachment strategy in which the library member was linked to the solid support by a photolabile linker and the tags were attached through a linker cleavable only by vigorous oxidation. Because the library members can be repetitively partially photoeluted from the solid support, library members can be utilized in multiple assays. Successive photoelution also permits a very high throughput iterative screening strategy: first, multiple beads are placed in 96-well microtiter plates; second, molecules are partially detached and transferred to assay plates; third, a metal binding assay identifies the active wells; fourth, the corresponding beads are rearrayed singly into new microtiter plates; fifth, single active molecules are identified; and sixth, the structures are decoded.

5.2. Screening Molecules for Binding to Targets

In the subject invention, candidate Lr molecules may be assayed by a variety of methods. Candidates which exhibit a desired level of binding affinity may be selected for further evaluation of their therapeutic effect, e.g., anti-infective efficacy, by using other assays such as transformed cell lines, primary cells in culture or animal models. For all the assays described herein, a single molecule, a mixture of them, or even an entire library of molecules may be assayed at once as appropriate. Also, more than one type of assay (or the same assay in series conducted under the same or different conditions) may be used to determine the therapeutic effect or other characteristics of a molecule of interest.

The molecules may be incubated with the entire target microorganism or a surface molecule therefrom. In certain embodiments, after adequate washing steps, the molecules may be eluted from the microbes and analyzed. In other embodiments, the affinity of a subject molecule for a surface molecule will be measured. In certain embodiments of the invention, those molecules that exhibit an affinity of at least 10⁻³ M will be selected. In certain embodiments of the invention, only hydrophilic molecules that bind a target will be selected so that uptake into cells is avoided.

In certain embodiments, the molecules will be pre-coupled to a particle. Binding may be in analyzed in this case by incubating a solution of a target molecule or microorganism with the particles, and quantifying the amount complexed to the target via an appropriate assay.

As a general matter, one or more inventive molecules may contacted with a target. Biological targets include, for example, enzymes, receptors, peptides, nucleic acid and the like. The biological target may be provided in the form of a purified or semi-purified composition, a cell lysate, a whole cell or tissue, or even a whole organism.

In certain of the subject assays, to evaluate the results using the subject compositions, comparisons may be made to known molecules, such as one with a known binding affinity for the target. For example, a known molecule and a new molecule of interest may be assayed. The result of the assay for the subject complex will be of a type and of a magnitude that may be compared to result for the known molecule. To the extent that the subject complex exhibits a type of response in the assay that is quantifiably different from that of the known molecule then the result for such complex in the assay would be deemed a positive or negative result. In certain assays, the magnitude of the response may be expressed as a percentage response with the known molecule result, e.g. 100% of the known result if they are the same.

Those skilled in the art will appreciate from the present description that the ability of said molecules to bind a target molecule or microorganism may be determined by using any of a variety of suitable assays. For example, in certain embodiments of the present invention, the ability of a candidate molecule to bind a target may be evaluated by an in vitro assay. Examples of assays contemplated for use in the present invention include, but are not limited to, variations of competitive binding assays, direct binding assays, and cell-based attachment assays. In certain embodiments, the full composition comprising the particle, linker and test molecule will be used. Assays to determine the efficacy of binding of a test molecule or subject composition may also be done in vivo. Such assays are well-known to one of skill in the art and, based on the present description, may be adapted to the methods of the present invention with no more than routine experimentation.

Any of the assays may be provided in kit format and may be automated. Many of the following particularized assays rely on general principles, such as blockage or prevention of transcription, that may apply to other particular assays. These teachings will also apply to assays of subject Lr molecules and libraries thereof.

All of the screening methods may be accomplished by using a variety of assay formats. In light of the present disclosure, those not expressly described herein will nevertheless be known and comprehended by one of ordinary skill in the art. Assay formats which approximate such conditions as formation of protein complexes or protein-nucleic acid complexes, and enzymatic activity may be generated in many different forms, as those skilled in the art will appreciate based on the present description and include but are not limited to assays based on cell-free systems, e.g. purified proteins or cell lysates, as well as cell-based assays which utilize intact cells.

A) Exemplary In Vitro Assays

As those skilled in the art will understand, based on the present description, binding assays may be used to detect agents that bind a target. Cell-free assays may be used to identify molecules that are capable of interacting with a target. In a preferred embodiment, cell-free assays for identifying such molecules are comprised essentially of a reaction mixture containing a target and a test molecule or a library of test molecules. A test molecule may be, e.g., a derivative of a known binding partner of the target, e.g., a biologically inactive peptide, or a small molecule. Agents to be tested for their ability to bind may be produced, for example, by bacteria, yeast or other organisms (e.g. natural products), produced chemically (e.g. small molecules, including peptidomimetics), or produced recombinantly. In certain embodiments, the test molecule is selected from the group consisting of lipids, carbohydrates, peptides, peptidomimetics, peptide-nucleic acids (PNAs), proteins, small molecules, natural products, aptamers and oligonucleotides. In other embodiments of the invention, the binding assays are not cell-free. In a preferred embodiment, such assays for identifying molecules that bind a target comprise a reaction mixture containing a target microorganism and a test molecule or a library of test molecules.

In many candidate screening programs which test libraries of molecules and natural extracts, high throughput assays are desirable in order to maximize the number of molecules surveyed in a given period of time. Assays of the present invention which are performed in cell-free systems, such as may be derived with purified or semi-purified proteins or with lysates, are often preferred as “primary” screens in that they may be generated to permit rapid development and relatively easy detection of binding between a target and a test molecule. Moreover, the effects of cellular toxicity and/or bioavailability of the test molecule may be generally ignored in the in vitro system, the assay instead being focused primarily on the ability of the molecule to bind the target. Accordingly, potential binding molecules may be detected in a cell-free assay generated by constitution of functional interactions of interest in a cell lysate. In an alternate format, the assay may be derived as a reconstituted protein mixture which, as described below, offers a number of benefits over lysate-based assays.

In one aspect, the present invention provides assays that may be used to screen for molecules that bind targets. In an exemplary binding assay, the molecule of interest is contacted with a mixture generated from target cell surface polypeptides. Detection and quantification of expected binding from to a target polypeptide provides a means for determining the molecule's efficacy at binding the target. The efficacy of the molecule may be assessed by generating dose response curves from data obtained using various concentrations of the test molecule. Moreover, a control assay may also be performed to provide a baseline for comparison. In the control assay, the formation of complexes is quantitated in the absence of the test molecule.

Complex formation between a molecule and a target molecule or microorganism may be detected by a variety of techniques, many of which are effectively described above. For instance, modulation in the formation of complexes may be quantitated using, for example, detectably labeled proteins (e.g. radiolabeled, fluorescently labeled, or enzymatically labeled), by immunoassay, or by chromatographic detection.

Accordingly, one exemplary screening assay of the present invention includes the steps of contacting a polypeptide or functional fragment thereof with a test molecule or library of test molecules and detecting the formation of complexes. For detection purposes, for example, the molecule may be labeled with a specific marker and the test molecule or library of test molecules labeled with a different marker. Interaction of a test molecule with a polypeptide or fragment thereof may then be detected by determining the level of the two labels after an incubation step and a washing step. The presence of two labels after the washing step is indicative of an interaction. Such an assay may also be modified to work with a whole target cell.

An interaction between a target and a molecule may also be identified by using real-time BIA (Biomolecular Interaction Analysis, Pharmacia Biosensor AB) which detects surface plasmon resonance (SPR), an optical phenomenon. Detection depends on changes in the mass concentration of macromolecules at the biospecific interface, and does not require any labeling of interactants. In one embodiment, a library of test molecules may be immobilized on a sensor surface, e.g., which forms one wall of a micro-flow cell. A solution containing the target is then flowed continuously over the sensor surface. A change in the resonance angle as shown on a signal recording, indicates that an interaction has occurred. This technique is further described, e.g., in BIAtechnology Handbook by Pharmacia.

In a preferred embodiment, it will be desirable to immobilize the target to facilitate separation of complexes from uncomplexed forms, as well as to accommodate automation of the assay. Binding of polypeptide to a test molecule may be accomplished in any vessel suitable for containing the reactants. Examples include microtitre plates, test tubes, and micro-centrifuge tubes. In one embodiment, a fusion protein may be provided which adds a domain that allows the target to be bound to a matrix. For example, glutathione-S-transferase/polypeptide (GST/polypeptide) fusion proteins may be adsorbed onto glutathione sepharose beads (Sigma Chemical, St. Louis, Mo.) or glutathione derivatized microtitre plates, which are then combined with a labeled test molecule (e.g., S³⁵ labeled, P³³ labeled, and the like, and the mixture incubated under conditions conducive to complex formation, e.g. at physiological conditions for salt and pH, though slightly more stringent conditions may be desired. Following incubation, the beads are washed to remove any unbound label, and the matrix immobilized and radiolabel determined directly (e.g. beads placed in scintillant), or in the supernatant after the complexes are subsequently dissociated. Alternatively, the complexes may be dissociated from the matrix, separated by SDS-PAGE, and the level of polypeptide or binding partner found in the bead fraction quantitated from the gel using standard electrophoretic techniques such as described in the appended examples. The above techniques could also be modified in which the test molecule is immobilized, and the labeled target is incubated with the immobilized test molecules. In one embodiment of the invention, the test molecules are immobilized, optionally via a linker, to a particle of the invention, e.g. to create the ultimate composition.

Other techniques for immobilizing targets or molecules on matrices may be used in the subject assays. For instance, a target or molecule may be immobilized utilizing conjugation of biotin and streptavidin. For instance, biotinylated polypeptide molecules may be prepared from biotin-NHS(N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with a target or molecule may be derivatized to the wells of the plate, and the target or molecule trapped in the wells by antibody conjugation. As above, preparations of test molecules are incubated in the polypeptide presenting wells of the plate, and the amount of complex trapped in the well may be quantitated. Exemplary methods for detecting such complexes, in addition to those described above for the GST-immobilized complexes, include immunodetection of complexes using antibodies reactive with the complex, or which are reactive with one of the complex components; as well as enzyme-linked assays which rely on detecting an enzymatic activity associated with a target or molecule, either intrinsic or extrinsic activity. In an instance of the latter, the enzyme may be chemically conjugated or provided as a fusion protein with the target or molecule. To illustrate, a target polypeptide may be chemically cross-linked or genetically fused with horseradish peroxidase, and the amount of polypeptide trapped in a complex with a molecule may be assessed with a chromogenic substrate of the enzyme, e.g. 3,3′-diamino-benzadine terahydrochloride or 4-chloro-1-napthol. Likewise, a fusion protein comprising the polypeptide and glutathione-S-transferase may be provided, and complex formation quantitated by detecting the GST activity using 1-chloro-2,4-dinitrobenzene (Habig et al (1974) J Biol Chem 249:7130).

For processes that rely on immunodetection for quantitating one of the components trapped in a complex, antibodies against a component, such as anti-polypeptide antibodies, may be used. Alternatively, the component to be detected in the complex may be “epitope tagged” in the form of a fusion protein which includes, in addition to the polypeptide sequence, a second polypeptide for which antibodies are readily available (e.g. from commercial sources). For instance, the GST fusion proteins described above may also be used for quantification of binding using antibodies against the GST moiety. Other useful epitope tags include myc-epitopes (e.g., see Ellison et al. (1991) J Biol Chem 266:21150-21157) which includes a 10-residue sequence from c-myc, as well as the pFLAG system (International Biotechnologies, Inc.) or the pEZZ-protein A system (Pharmacia, N.J.).

In certain in vitro embodiments of the present assay, the solution containing the target comprises a reconstituted protein mixture of at least semi-purified proteins. By semi-purified, it is meant that the components utilized in the reconstituted mixture have been previously separated from other cellular or viral proteins. For instance, in contrast to cell lysates, a target protein is present in the mixture to at least 50% purity relative to all other proteins in the mixture, and more preferably are present at 90-95% purity. In certain embodiments of the subject method, the reconstituted protein mixture is derived by mixing highly purified proteins such that the reconstituted mixture substantially lacks other proteins (such as of cellular or viral origin) which might interfere with or otherwise alter the ability to measure binding activity. In one embodiment, the use of reconstituted protein mixtures allows more careful control of the target:molecule interaction conditions.

In still other embodiments of the present invention, variations of convention cell-cell attachment (or “adherence”) assays may be utilized in order to determine the ability of a test molecule or composition of particle attached to a test molecule to bind a target microorganism, and prevent it from binding to other cells. For example, the ability of a target microorganism to bind HeLa, Kato-3, HEp-2, or other suitable cell lines may be assayed in the presence of said test molecule or composition. If cell-cell adherence is prevented, then the molecule or composition may be useful as a therapeutic agent. An adherence assay utilizing normal physiological flora could optionally be performed along with the assay utilizing the target microorganism. A molecule or composition that prevents adherence by the target microorganism but not by normal flora would be preferentially selected over one that prevented adherence by both. Exemplary attachment assays have been described in Baldini, M. M., et al, Infect Immun (1986) 52(1):334-6; Scaletsky, I C, et al. Infect Immun (1984) 45(2):534-6; and Chimiela, M, et al., J Physiol Pharmacol (1997) 48(3):393-404. Such assays could be varied for use in the present invention without undue experimentation by one of skill in the art.

Assaying binding resulting from a given target:molecule interaction may be accomplished in any vessel suitable for containing the reactants. Examples include microtitre plates, test tubes, and micro-centrifuge tubes.

B) Production of Target Molecules for Use in Assays

A target molecule may be expressed on the surface of an engineered cell, either pathogenic or not, and molecules screened for binding to it using any of the assays described above. Likewise, a target molecule may be produced in an engineered cell, and isolated for use with the assays described above. Methods for producing such recombinant reagent cells are well-known in the art. For example, ligating a polynucleotide coding sequence into a gene construct, such as an expression vector, and transforming or transfecting into hosts, either eukaryotic (yeast, avian, insect or mammalian) or prokaryotic (bacterial cells), are standard procedures used in producing other well-known proteins, including sequences encoding exogenous receptor and peptide libraries. Similar procedures, or modifications thereof, can be employed to prepare recombinant reagent cells of the present invention by tissue-culture technology in accord with the subject invention.

In general, it will be desirable that the vector be capable of replication in the host cell. It may be a DNA which is integrated into the host genome, and thereafter is replicated as a part of the chromosomal DNA, or it may be DNA which replicates autonomously, as in the case of a plasmid. In the latter case, the vector will include an origin of replication which is functional in the host. In the case of an integrating vector, the vector may include sequences which facilitate integration, e.g., sequences homologous to host sequences, or encoding integrases.

Representative examples of vectors which may be used include viral vectors, phage, plasmids, phagemids, cosmids, phosmids, mammalian artificial chromosomes (MAC), bacterial artificial chromosomes (BACs), bacteriophage P1, P1-based artificial chromosomes (PACs), yeast artificial chromosomes (YACs), yeast plasmids, and any other vectors suitable for a specific host cell and capable of stably maintaining and expressing a genomic DNA insert of at least 20 kb, and more preferably greater than 50-75 kb.

Appropriate cloning and expression vectors for use with bacterial, fungal, yeast, and mammalian cellular hosts are known in the art, and are described in, for example, Powels et al. (Cloning Vectors: A Laboratory Manual, Elsevier, New York, 1985). Mammalian expression vectors may comprise non-transcribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a poly-adenylation site, splice donor and acceptor sites, and transcriptional termination sequences.

Certain mammalian expression vectors contain both prokaryotic sequences, to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papillomavirus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press: 1989) Chapters 16 and 17.

Preferred vectors for the present invention are the so-called artificial chromosomes. One feature of these vectors is their ability to carry large genetic inserts, e.g., greater than 50 kb, with enough mitotic and meiotic stabilities to make their genetic manipulation straightforward. The upper limit on the size of the insert is often great enough that thousands of genes can be included on one vector. Thus, a single vector could provide the coding sequences for hundreds of GPCRs.

In certain preferred embodiments, the vector is a mammalian artificial chromosome, e.g., which can stably incorporate the coding sequences for at least five diferrent GCPRs. Exemplary MACs are described in, for example, U.S. Pat. Nos. 5,721,118 and 6,077,697, as well as Csonka et al. J Cell Sci. (2000) 113:3207-3216; Ebersole et al. Hum Mol Genet (2000) 9:1623-31; deJong et al. Cytometry (1999) 35:129-133; and Schindelhauer, Bioessays (1999) 21(1):76-83.

Another artificial chromosome which can be adapted for use in the present invention is the baculovirus artificial chromosomes, such as described in detail in U.S. Pat. No. 6,090,584. That patent discloses a baculovirus artificial chromosome which has the lef-8 gene inactivated. The baculovirus artificial chromosome allows the cloning and expression of heterologous genes in insect and mammalian cells.

P1-based artificial chromosomes (PACs) and bacterial artificial chromosomes (BACs) have significantly expanded the size of fragments from eukaryotic genomes that can be stably cloned in E. coli and the like as plasmid molecules. Advantages of these system include the low copy number of the vector (based on the single copy F plasmid of E. coli), large possible insert size (clones containing inserts of up to 300 Kb have been propagated), stability of clones in vivo, high cloning efficiency, and easy manipulation of clones by standard techniques (Shizuya et al. (1992) PNAS 89:8794-8797). The BAC and PAC systems provide a method to construct a stable library of large inserts, which in certain instances can be critical to the success of the subject method.

The transcriptional and translational control sequences in expression vectors to be used in transforming mammalian cells may be provided by viral sources. For example, commonly used promoters and enhancers are derived from Polyoma, Adenovirus 2, Simian Virus 40 (SV40), and human cytomegalovirus. DNA sequences derived from the SV40 viral genome, for example, SV40 origin, early and late promoter, enhancer, splice, and polyadenylation sites may be used to provide the other genetic elements required for expression of a heterologous DNA sequence. The early and late promoters are particularly useful because both are obtained easily from the virus as a fragment which also contains the SV40 viral origin of replication (Fiers et al. Nature (1978) 273:111) Smaller or larger SV40 fragments may also be used, provided the approximately 250 bp sequence extending from the Hind III site toward the Bgl I site located in the viral origin of replication is included. Exemplary vectors can be constructed as disclosed by Okayama and Berg, Mol. Cell Biol (1983) 3:280. A useful system for stable high level expression of mammalian receptor cDNAs in C127 murine mammary epithelial cells can be constructed substantially as described by Cosman et al, Mol. Immunol. (1986) 23:935. Other expression vectors for use in mammalian host cells are derived from retroviruses.

In other embodiments, the use of viral transfection can provide stably integrated copies of the expression construct. In particular, the use of retroviral, adenoviral or adeno-associated viral vectors is contemplated as a means for providing a stably transfected cell line which expresses an exogenous receptor, and/or a polypeptide library.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al. (1983) in Experimental Manipulation of Gene Expression, ed. M. Inouye Academic Press, p. 83, incorporated by reference herein). These vectors can replicate in E. coli due the presence of the pBR322 ori, and in S. cerevisiae due to the replication determinant of the yeast 2 micron plasmid. In addition, drug resistance markers such as ampicillin can be used. Moreover, if yeast are used as a host cell, it will be understood that the expression of a gene in a yeast cell requires a promoter which is functional in yeast. Suitable promoters include the promoters for metallothionein, 3-phosphoglycerate kinase (Hitzeman et al., J. Biol. Chem. 255, 2073 (1980) or other glycolytic enzymes (Hess et al., J. Adv. Enzyme Req. 7, 149 (1968); and Holland et al. Biochemistry 17, 4900 (1978)), such as enolase, glyceraldehyde-3-phosphate dehydrogenase, hexokinase, pyruvate decarboxylase, phospho-fructokinase, glucose-6-phosphate isomerase, 3-phosphoglycerate mutase, pyruvate kinase, triosephosphate isomerase, phospho-glucose isomerase, and glucokinase. Suitable vectors and promoters for use in yeast expression are further described in R. Hitzeman et al., EPO Publn. No. 73,657. Other promoters, which have the additional advantage of transcription controlled by growth conditions, are the promoter regions for alcohol dehydrogenase 2, isocytochrome C, acid phosphatase, degradative enzymes associated with nitrogen metabolism, and the aforementioned metallothionein and glyceraldehyde-3-phosphate dehydrogenase, as well as enzymes responsible for maltose and galactose utilization. Finally, promoters that are active in only one of the two haploid mating types may be appropriate in certain circumstances.

In some instances, it may be desirable to derive the host cell using insect cells. In such embodiments, recombinant polypeptides can be expressed by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

Libraries of random peptides or cDNA fragments may be expressed in many ways, including as portions of chimeric proteins. As described below, where secretion of the peptide. library is desired, the peptide library can be engineered for secretion or transport to the extracellular space via the yeast pheromone system In constructing suitable expression plasmids, the termination sequences associated with these genes, or with other genes which are efficiently expressed in yeast, may also be ligated into the expression vector 3′ of the heterologous coding sequences to provide polyadenylation and termination of the mRNA.

The vector should, as pointed out above, include at least one origin of replication for the host cell into which the vector is to be transfected. If also necessary, the vector can include one or more copy-control sequence for controlling the number of copies of the vector in any one cell.

The vector is transfected into and propagated in the appropriate host. Methods for transfecting the host cells with the vector can be readily adapted from procedures that are known in the art. For example, the vector can be introduced into the host cell by such techniques as the use electroporation, precipitation with DEAE-Dextran or calcium phosphate, or lipofection.

C. In Vivo Assays

The most promising molecules and compositions selected from the in vitro analysis may be tested in such animals models for prophylactic and therapeutic efficacy. Animal models exist for most of the diseases caused by the target microorganisms, which may be used for in vivo analysis of the compositions of the present invention. For example, such animals may be genetically altered to be predisposed to a more serious form of gastrointestinal disease caused by a target microorganism. Other animals may be simply normal animals infected with a target microorganism. The composition may be analyzed for its ability to clear an infection or prevent an infection in the animals by the target microorganism of interest, as well as for any potential side effects.

Non-limiting examples of animals models contemplated for use in the present invention include clindamycin pretreated golden Syrian hamsters challenged with toxin-producing C. difficile, healthy and immunocompromised mice infected with vancomycin-resistant enterococcus faecium, H. pylori-infected C57BL/6 mice, immunosuppressed BALB/c mice infected with oropharyngeal candidiasis, and the like.

6. Targets

In certain embodiments of the present invention, the compositions target those microorganisms capable of producing a disease of or infection in the gastrointestinal tract. In one embodiment, the compositions prevent the adherence of such microorganisms to the gastrointestinal tract, thus inhibiting the development of disease. In another embodiment, the compositions bind and clear the microorganisms from the gastrointestinal tract. In this embodiment, the compositions may reduce the time such microorganisms are in the gastrointestinal tract and/or reduce their population in the tract. Any one or subset of the surface molecules on the target microorganism may be used to bind the microorganism to the compositions of the present invention. In still other embodiments, the compositions may kill the target microorganism. The ultimate goal of the invention is the decolonization of or prevention of colonization by the gastrointestinal microorganisms in the patient, thus curing or preventing an infection.

Exemplary genera and species of bacteria that may be targeted by the compositions of the present invention in the treatment of a gastrointestinal disease or infection include Clostridium, Vancomycin-resistant Enterococcus, Helicobacter pylori, Campylobacter, Salmonella non-typhoid, enterohemorrhagic E. coli (EHEC), enteropathogenic E. coli (EPEC), enterotoxigenic E. coli (ETEC), enteroaggregative E. coli, Shigella, Vibrio cholerae, Staphylococcus, Streptococcus, Yersinia, Listeria, Bacillus cereus, Bacillus anthracis, and Francisella tularensis.

Exemplary genera and species of viruses that may be targeted by the compositions of the present invention in the treatment of a gastrointestinal disease or infection include Rotavirus, Norwalk virus, Astrovirus, Hepatitis B, and Human Immunodeficiency Virus.

Exemplary genera and species of parasites that may be targeted by the compositions of the present invention in the treatment of a gastrointestinal disease or infection include Plasmodium, Giardia, Cryptosporidium, Entamoeba, and Cylospora cayetanensis.

Exemplary genera and species of fungi that may be targeted by the compositions of the present invention in the treatment of a gastrointestinal disease or infection include Histoplasmas, Blastomycetes, Coccidioides, Paracoccidioides, Aspergillus and Candida.

In certain embodiments, the compositions of the present invention target microbial infection in other orifices in the body, for example, a vaginal or oral fungal infection. Such compositions may act in a mode similar to that of those designed to target gastrointestinal microorganisms, and may be cleared from the orifice.

Exemplary genera and species of bacteria that may be targeted by the compositions of the present invention in treating infection of orifices include Gardnerella vaginalis, L. acidophilus, Staphylococci, Bacteroides, Hemophilis vaginalis, Bacteroides, Mobiluncus, Mycoplasma hominis, Chlamydia trachomatis, Mycoplasma hominis, Ureaplasma urealyticum, alpha- and beta-hemolytic Streptrococcus, Streptococcus salivarius and mutans, Neisseria, Hemophilus, Pneumococci, Bordetella, Corynebacterium and Gonorrhea.

Exemplary genera and species of viruses that may be targeted by the compositions of the present invention in treating infection of orifices include herpes simplex and human papillomavirus.

Exemplary genera and species of parasites that may be targeted by the compositions of the present invention in treating infection of orifices include Trichomonas vaginalis and Mycoplasmas.

Exemplary genera and species of fungi that may be targeted by the compositions of the present invention in treating infection of orifices include Histoplasmas, Blastomycetes, Coccidioides, Paracoccidioides, Candida, Torulopsis, and Aspergillus.

7. Pharmaceutical Compositions

The present invention provides pharmaceutical formulations of the compositions of the invention. In part, the subject invention is directed to formulations of compositions able to release a biologically active agent. In another aspect, the subject compositions may be used in the manufacture of a medicament for any number of uses, including, for example, treating any disease or other treatable condition of a patient.

The pharmaceutical compositions of the present invention may be administered by various means, depending on their intended use, as is well known in the art. For example, if compositions of the present invention are to be administered orally, they may be formulated as tablets, capsules, granules, powders or syrups. Alternatively, formulations of the present invention may be suppositories. These formulations may be prepared by conventional means, and, if desired, the compositions may be mixed with any conventional additive, such as an excipient, a binder, a disintegrating agent, a lubricant, a corrigent, a solubilizing agent, a suspension aid, an emulsifying agent or a coating agent.

In formulations of the subject invention, wetting agents, emulsifiers and lubricants, such as sodium lauryl sulfate and magnesium stearate, as well as coloring agents, release agents, coating agents, sweetening, flavoring and perfuming agents, preservatives and antioxidants may be present in the formulated agents.

Subject compositions may be suitable for oral, rectal and/or vaginal administration. The formulations may conveniently be presented in unit dosage form and may be prepared by any methods well known in the art of pharmacy. The amount of agent that may be combined with a carrier material to produce a single dose vary depending upon the subject being treated, and the particular mode of administration.

Methods of preparing these formulations include the step of bringing into association agents of the present invention with the carrier and, optionally, one or more accessory ingredients. In general, the formulations are prepared by uniformly and intimately bringing into association agents with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a molecule thereof as an active ingredient. Compositions of the present invention may also be administered as a bolus, electuary, or paste.

In solid dosage forms for oral administration (capsules, tablets, pills, dragees, powders, granules and the like), the particle is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium molecules; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the supplement or components thereof moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Liquid dosage forms for oral administration include pharmaceutically acceptable emulsions, microemulsions, solutions, suspensions, syrups and elixirs. In addition to the compound, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Suspensions, in addition to compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a particle of the present invention with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent. Formulations which are suitable for vaginal administration also include pessaries, tampons, creams, gels, pastes, foams or spray formulations containing such carriers as are known in the art to be appropriate.

Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

8. Therapeutic Methods

In another aspect, the present invention is directed to methods of using the subject compositions for prophylactic or therapeutic treatment of a colonization or infection of microorganisms in a patient. In some embodiments of the invention, said colonization is gastrointestinal. In embodiments where the treatment is therapeutic, said treatment may result in the decolonization of the microorganisms. In certain embodiments, use of the subject that release a biologically active agent in a sustained manner allows for different treatment regimens than are possible with other modes of administration of such therapeutic agents.

The dosage of any compound of the present invention will vary depending on the symptoms, age and body weight of the patient, the nature and severity of the disorder to be treated or prevented, the route of administration, and the form of the supplement. Any of the subject formulations may be administered in a single dose or in divided doses. Dosages for the compounds of the present invention may be readily determined by techniques known to those of skill in the art or as taught herein. Also, the present invention contemplates mixtures of more than one subject compound, as well as other therapeutic agents.

An effective dose or amount, and any possible affects on the timing of administration of the formulation, may need to be identified for any particular compound of the present invention. This may be accomplished by routine experiment as described herein, using one or more groups of animals (preferably at least 5 animals per group), or in human trials if appropriate. The effectiveness of any compound and method of treatment or prevention may be assessed by administering the composition and assessing the effect of the administration by measuring one or more indices associated with the infection of interest, and comparing the post-treatment values of these indices to the values of the same indices prior to treatment.

The precise time of administration and amount of any particular compound that will yield the most effective treatment in a given patient will depend upon the activity, pharmacokinetics, and bioavailability of a particular compound, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more of the relevant indices at predetermined times during a 24-hour period. Treatment, including supplement, amounts, times of administration and formulation, may be optimized according to the results of such monitoring. The patient may be periodically reevaluated to determine the extent of improvement by measuring the same parameters, the first such reevaluation typically occurring at the end of one week from the onset of therapy, and subsequent reevaluations occurring every one to two weeks during therapy and then every month thereafter. Adjustments to the amount(s) of agent administered and possibly to the time of administration may be made based on these reevaluations.

Treatment may be initiated with smaller dosages which are less than the optimum dose of the compound. Thereafter, the dosage may be increased by small increments until the optimum therapeutic effect is attained.

The combined use of several compositions of the present invention, or alternatively compositions with multiple binding moieties, may reduce the required dosage for any individual component because the onset and duration of effect of the different components may be complimentary. In such combined therapy, the different active agents may be delivered together or separately, and simultaneously or at different times within the day.

Toxicity and therapeutic efficacy of subject compositions able to release biologically active agents may be determined by standard pharmaceutical procedures in cell cultures or experimental animals, e.g., for determining the LD₅₀ and the ED₅₀. Compositions that exhibit large therapeutic indices are preferred. Although compounds that exhibit toxic side effects may be used, e.g. an agent released from the subject compositions, care should be taken to design a delivery system that targets the compounds to the desired site in order to reduce side effects. Furthermore, if a subject composition releases a biologically active agent, it will be important to ensure that the agent does not reduce the efficacy of the composition in binding and clearing the target microorganism.

The data obtained from the cell culture assays and animal studies may be used in formulating a range of dosage for use in humans. The dosage of any supplement, or alternatively of any components therein, lies preferably within a range of circulating concentrations that include the ED₅₀ with little or no toxicity. The dosage may vary within this range depending upon the dosage form employed and the route of administration utilized. For agents of the present invention, the therapeutically effective dose may be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information may be used to more accurately determine useful doses in humans. Levels in plasma may be measured, for example, by high performance liquid chromatography.

9. Diagnostics

In another aspect, the present invention is directed to methods of using the subject compositions for diagnosis of a colonization or infection of microorganisms in a patient. In one embodiment of the present invention, subject compositions able to bind a gastrointestinal microorganism can be administered to a patient. After the compositions are cleared from the patient, they may be collected and evaluated for bound microorganisms. The presence of bound microorganism may indicate an infection by that microorganism. In another embodiment of the present invention, subject compositions able to bind a gastrointestinal microorganism can be administered to a patient throughout the course of treatment for an infection by the gastrointestinal microorganism of interest. After the compositions are cleared from the patient, they may be collected and quantitatively evaluated for bound microorganisms. A decreased quantity of bound microorganism may indicate that the treatment of the infection is effective. In still another embodiment of the invention, the ligands on a composition able to bind a target gastrointestinal microorganism may undergo a physical change when bound to a microorganism, e.g. a colorimetric change, so that when cleared from the patient, the presence of bound microorganisms may be detected by the color of the cleared particles.

10. Kits

The invention further provides kits for use in treating or diagnosing a disease or condition. For example, the kit may comprise a pharmaceutical formulation of a non-absorbable composition of the present invention. The formulation may be packaged in a suitable container. The kit may further comprise instructions for using the kit. In one embodiment, the kit comprises a pharmaceutical formulation of a subject composition able to release a biological agent and instructions for use.

Kit components may be packaged for either manual or partially or wholly automated practice of the foregoing methods. In other embodiments involving kits, this invention contemplates a kit including compositions of the present invention, and optionally instructions for their use. Such kits may have a variety of uses, including, for example, imaging, diagnosis, therapy, and other applications.

### REFERENCES

The contents of all cited references including literature references, issued patents, published or non-published patent applications cited throughout this application as well as those listed below are hereby expressly incorporated by reference in their entireties. In case of conflict, the present application, including any definitions herein, will control. Russell, A. D., J. Appl. Microbiol. (1997) 83:155-165;

Equivalents

The present invention provides among other things compositions for treating gastrointestinal or other disorders, methods of treating disorders using such compositions and kits comprising such compositions. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

All publications and patents mentioned herein are hereby incorporated by reference in their entirety as if each individual publication or patent was specifically and individually indicated to be incorporated by reference. In case of conflict, the present application, including any definitions herein, will control.

We claim:

1. A non-absorbable composition comprised of a particle having a formula selected from the group consisting of: 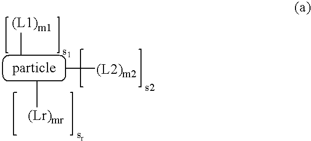
wherein;
L1, L2 . . . Lr, each independently, represent a molecule that is able to bind or kill a gastrointestinal or other microorganism; wherein said molecule is selected from the group consisting of lipids, carbohydrates, peptides, peptidomimetics, peptide-nucleic acids (PNAs), proteins, small molecules, natural products, aptamers and oligonucleotides; and wherein each Lr optionally may be conjugated to said particle through a linker molecule;
mr, independently for each Lr, is at least 1;
sr, independently for each Lr, is at least 1;
and at least 2 different Lr are conjugated to said particle; 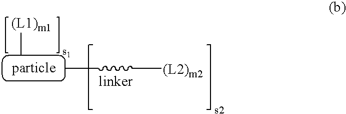
wherein;
L1 and L2 each independently, represent a molecule that is able to bind or kill a gastrointestinal or other microorganism; wherein said molecule is selected from the group consisting of lipids, carbohydrates, peptides, peptidomimetics, peptide-nucleic acids (PNAs), proteins, small molecules, natural products, aptamers and oligonucleotides; wherein L1 optionally may be conjugated to said particle through a linker molecule of the same type or a different type than the linker through which L2 is conjugated to said particle;
mr, independently for each Lr, is at least 1; and
sr, independently for each Lr, is at least 1; 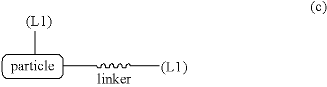
wherein;
L1 represents a molecule that is able to bind or kill a gastrointestinal or other microorganism; wherein said molecule is selected from the group consisting of lipids, carbohydrates, peptides, peptidomimetics, peptide-nucleic acids (PNAs), proteins, small molecules, natural products, aptamers and oligonucleotides; wherein some L1 optionally may be conjugated to said particle through a linker molecule of a different type than the linker through which other L1 are conjugated to said particle; and
at least two different L1-particle linkages are present; 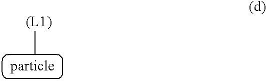
wherein;
L1 represents a molecule that is able to bind or kill a gastrointestinal or other microorganism; wherein said molecule is selected from the group consisting of lipids, carbohydrates, peptides, peptidomimetics, peptide-nucleic acids (PNAs), proteins, small molecules, natural products, aptamers and oligonucleotides; wherein L1 optionally may be conjugated to said particle through a linker molecule; and
said particle is able to release biologically active agents, contains agents able to kill a target microorganism, or is otherwise able to aid in the binding or killing of the target microorganism; 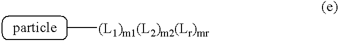
wherein;
L1, L2 . . . Lr, each independently, represent a molecule that is able to bind or kill a gastrointestinal or other microorganism; wherein said molecule is selected from the group consisting of lipids, carbohydrates, peptides, peptidomimetics, peptide-nucleic acids (PNAs), proteins, small molecules, natural products, aptamers and oligonucleotides; wherein multiple Lr are conjugated to the particle through a single point of attachment, optionally through a linker molecule, and may be arranged in any order;
r is at least 2; and
mr, independently for each Lr, is at least 1; and 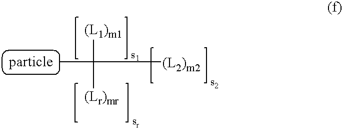
wherein;
L1, L2 . . . Lr, each independently, represent a molecule that is able to bind or kill a gastrointestinal or other microorganism; wherein said molecule is selected from the group consisting of lipids, carbohydrates, peptides, peptidomimetics, peptide-nucleic acids (PNAs), proteins, small molecules, natural products, aptamers and oligonucleotides;
r is at least 2;
s2, independently for each Lr, is at least 1; and
mr, independently for each Lr, is at least 1.

2. The composition of claim 1, wherein said particle is comprised of at least one polymer selected from the group consisting of polystyrene, polymethylmethacrylate, polyethylene glycol, polypropylene, polycarbonate, polyethylene, polyurethane, polypropylene glycol, expanded polytetrafluoroethylenes, fluorinated ethylene propylene, polyvinylalcohol, and polycarbonate.

3. The composition of claim 1, wherein said particle is comprised of a controlled release polymer.

4. The composition of claim 3, wherein said particle contains a biologically active agent.

5. The composition of claim 1, wherein said particle is comprised of a polymer and is a bead.

6. The composition of claim 5, wherein said bead has a diameter in the range of about 1 to about 50 microns.

7. The composition of claim 5, wherein said polymer is polystyrene.

8. The composition of claim 1, wherein said particle is a macromolecular polymeric molecule.

9. The composition of claim 1, wherein said linker is a linear polymer.

10. The composition of claim 1, wherein said linker for each occurrence may be selected from the group consisting of: a linear polymer and a branched polymer.

11. The composition of claim 9, wherein said linear polymer is comprised of at least one of polyethylene glycol or polypropylene.

12. The composition of claim 9, wherein said linear polymer is a polypeptide.

13. The composition of claim 12, wherein said linker is polyglycine.

14. The composition of claim 1, wherein said microorganism is capable of causing a gastrointestinal infection or disease and is selected from the group consisting of: bacteria, viruses, fungi, and parasites.

15. The composition of claim 1, wherein the composition has formula (a), (b), (e), or (f), and at least one Lr is a molecule able to bind a microorganism, and at least one other Lr is a molecule able to kill a microorganism.

16. The composition of claim 1, wherein the composition has formula (a), (b), (e), or (f), and wherein r is 2 and L1 is a molecule able to bind a microorganism, and L2 is a molecule able to kill a microorganism.

17. The composition of claim 1, wherein each Lr is able to bind the same microorganism.

18. The composition of claim 1, wherein the composition has formula (a), (b), (e), or (f) and each Lr binds a different microorganism.

19. A method of treating or diagnosing an establishment of microorganisms in a patient comprising administration of a therapeutically effective amount of a pharmaceutical composition that comprises any of the non-absorbable compositions of claim 1 to a patient.

20. A kit comprising a pharmaceutical composition that comprises a non-absorbable composition of claim 1, and instructions for using said pharmaceutical composition.
